(19) United States
(12) Patent Application Publication
(10) Pub. No.: US 2012/0277659 A1
(43) Pub. Date: Nov. 1, 2012

(54) SENSOR-LOTION SYSTEM FOR USE WITH BODY TREATMENT DEVICES

(75) Inventors: Ilya Yaroslavsky, North Andover, MA (US); Gregory B. Altshuler, Lincoln, MA (US); Mikhail Z. Smirnov, Burlington, MA (US); David Tabatadze, Worcester, MA (US); Oldrich M. Laznicka, JR., Wellesley, MA (US)

(73) Assignee: PALOMAR MEDICAL TECHNOLOGIES, INC., Burlington, MA (US)

(21) Appl. No.: 13/460,418

(22) Filed: Apr. 30, 2012

Related U.S. Application Data
(60) Provisional application No. 61/480,890, filed on Apr. 29, 2011.

(51) Int. Cl. A61M 37/00

(52) U.S. Cl. 604/20

(57) ABSTRACT

Controls improve skin and/or eye safety for use of a light based photocosmetic device. The sensors having high spatial resolution and the low probability of sensor failure and improve skin and/or eye safety by differentiating safe and unsafe firing conditions. The system and/or the device is able to identify a topical present on the skin due to characteristics indicative of that topical that are sensed by the system. The topical can be identified by, for example, impedance level, marker(s), and/or multiple characteristics in a multi-phase system. The sensor(s) can improve safety by checking the presence of contact and the uniformity of contact with the identified topical throughout the treatment cycle.

### PRIORITY

This application claims the benefit of and priority to U.S. Ser. No. 61/480,890, filed Apr. 29, 2011 and entitled “Sensor-lotion System for Use with Body Treatment Devices,” the contents of which are incorporated by reference in its entirety.

### BACKGROUND

Use of directed energy (electromagnetic, acoustic, etc.) is becoming a technique of choice for the treatment of a number of medical, hygienic, and cosmetic conditions. Light in the wavelength range between 380 nm and 10000 nm is often used. Relevant examples include treatments of skin conditions (e.g., acne vulgaris) or use in oral hygiene (e.g., for treatment and prevention of periodontal disease).

Device implementations allowing self-application by a user and oriented towards home use are of particular interest. However, combining sufficient efficacy and high safety in a hand-held, consumer-use, low-cost device is a challenging task, which has not been adequately resolved so far. Providing the safe operation of laser and pulsed light medical devices remains a difficult problem. Most systems of this type include contact sensors designed to stop or prevent firing if the unsafe conditions occur. While contact sensors have received a lot of attention in the past years, existing sensors are not reliable enough at differentiating safe and unsafe firing conditions.

Detection of some unsafe conditions requires sensors that analyze the treatment area with a high spatial resolution. For instance, firing laser and pulsed light devices into an open eye must be prevented and/or avoided even though the exposed area of the eye tissue is very small. Combining high spatial resolution with the low probability of sensor failure is a challenging task.

### SUMMARY

In accordance with the improvements disclosed herein, in one embodiment a topical compound such as a lotion is used in conjunction with an apparatus including a sensor to ensure safe delivery of electromagnetic energy only to areas of the human body designated for treatment.

In one aspect the disclosure relates to a photocosmetic device including a handpiece having a source for generating energy for application to tissue. The handpiece has a distal end through which the energy can be applied to tissue. At least one sensor is coupled to the handpiece and is adapted to generate a signal in response to detecting contact between at least a portion of the distal end of the handpiece and a topical substance disposed over a portion of the tissue where treatment is desired. A feedback mechanism is in communication with the sensor. The energy source is activated in response to receiving the signal in response to detecting contact (e.g., the detection signal). Optionally, the handpiece has a scan mechanism for directing the energy to different portions of the tissue.

In some embodiments, the feedback mechanism is adapted to deactivate the energy source subsequent to its activation in response to absence of a detection signal from the sensor. The feedback mechanism may be in communication with the scan mechanism so as to activate the scan mechanism in response to receiving the detection signal from the sensor. The scan mechanism may be adapted to direct the radiation to the different tissue portions subsequent to its initial activation based on a predetermined protocol.

The photocosmetic device can further include a mechanism to differentiate between signals from a topical substance disposed over a portion of the tissue and from a bulk volume of the topical substance. For example, the bulk volume of the topical substance over a tissue is so thick a volume that the presence of the portion of tissue is undeterminable. Alternatively, the bulk volume of the topical substance is a thick “blob” of the topical substance disposed on the device (e.g., the handpiece) and is not in contact with the tissue.

In another aspect, the disclosure relates to a photocosmetic device having a handpiece including a source for generating energy for application to tissue and at least one detector coupled to the handpiece for detecting a topical substance disposed on the tissue when placed in proximity of the topical substance. The photocosmetic device also includes a feedback mechanism in communication with the source and the detector. The feedback mechanism activates the source in response to detection of the topical substance on the tissue by the detector. The feedback mechanism may be adapted to deactivate the source subsequent to activation in response to a signal from the detector indicating absence of said topical substance on the tissue.

In another aspect, the disclosure relates to a photocosmetic device having a source for generating electromagnetic radiation, a radiation transmission path for transmitting the radiation from the source to a radiation transmissive optical window through which the radiation can be applied to the skin, where the optical window has a perimeter adapted for positioning over the skin. The device includes a plurality of sensors for detecting the presence of a topical substance over the skin. The sensors are positioned relative to the window such that each sensor is capable of determining whether a selected portion of said perimeter is in contact with or in proximity to the topical substance disposed over the skin. The device includes a feedback mechanism in communication with the sensors and the radiation source and the feedback mechanism deactivates the radiation source if at least one of the sensors indicates absence of contact between a respective portion of the perimeter and the topical substance disposed over the skin.

In another aspect, the disclosure relates to a photocosmetic device including a handpiece adapted for positioning in proximity of tissue at a distal end thereof. The handpiece includes an optical path for transmitting energy from an energy source to the distal end for application to the tissue, at least a sensor coupled to the handpiece for generating a signal indicative of presence of a selected topical substance on the tissue in proximity of the distal end and a feedback mechanism in communication with the sensor and the source. The feedback mechanism activates the source in response to receiving the signal from the sensor. In some embodiments, the feedback mechanism is adapted to deactivate the source subsequent to its activation in absence of the signal from the sensor.

In another aspect, the disclosure relates to a photocosmetic device having a frame adapted for positioning in proximity of tissue to define an area of the tissue, a source for generating optical energy, and a scan mechanism coupled to the source for moving the source so as to apply optical energy to different portions of the area of the tissue. The photocosmetic device has a sensor adapted for detecting presence of a topical lotion on the tissue, the sensor generates a signal in response to detection of the topical lotion on the tissue. The photocosmetic device has a feedback mechanism in communication with the scan mechanism and the sensor. The feedback mechanism triggers the scan mechanism to effect the movement of the source in response to receiving the signal from the sensor.

In another aspect the disclosure relates to a photocosmetic device adapted for application of optical energy to tissue the device having a sensor adapted for detecting a topical substance in contact with the tissue and a control mechanism in communication with the sensor. The control mechanism permits application of the optical energy to a tissue portion only if the sensor detects the topical substance on said tissue portion. The photocosmetic device may be, for example, a hand piece. In some embodiments, the device also includes a source for generating said optical energy. The control mechanism can optionally cause a transition of the source from a de-activated state to an activated state in response to detection of the topical substance on the tissue by the sensor. The control mechanism can optionally maintain the source in an activated state subsequent to its initial activation if the sensor continues to detect the topical substance on the tissue. The sensor may, for example, detect impedance or a signature in an impedance curve indicative of detection of the topical substance on the tissue.

In another aspect, the disclosure relates to a system for treating tissue including a hand piece having an energy source configured to deliver energy to a tissue surface, a topical substance configured to be applied to the tissue surface, and a recognition mechanism. The recognition mechanism is in communication with the energy source and is configured to allow activation of the energy source in response to recognition of the topical substance on at least one recognition site of the tissue surface. The topical substance may be, for example, a ferromagnetic substance. Optionally, the topical substance includes at least one tag defining at least one characteristic configured to be recognized by the recognition mechanism. In one embodiment, the recognition mechanism is configured to distinguish the topical substance having the at least one tag from another topical substance having the at least one tag. In another embodiment, the recognition mechanism is configured to distinguish the topical substance having the at least one tag from the topical substance having at least one alternative tag. The recognition mechanism can include a sensor in communication with the at least one recognition site of the tissue surface. The sensor may be coupled to any of a number of suitable surfaces on the system such as, for example, the hand piece or the energy emitter. In one embodiment, the sensor includes a light emitter and a detector. The sensor may be configured to determine a parameter of the recognition site determinative of a presence or absence of the topical substance. In one embodiment, the recognition mechanism is measurement of impedance in a pre-defined set of discrete frequencies and computing a set of recognition parameters from the impedance measurements and determining whether these recognition parameters are within the desired area of the parameter space.

In one embodiment, the topical substance is a multi-phase system and at least a first and a second phase of the multi-phase system each contribute to a signal indicative of the presence of the topical substance. In some embodiments, the first phase is a background solution and the second phase is at least one particle suspended in the background solution. In other embodiments, the multi-phase system includes two or more distinct active components. The multi-phase system may include conductive particles suspended in a dielectric solution. The multi-phase system may include a ferromagnetic substance suspended in a dielectric solution. The multi-phase system may include at least one layered tag with at least one of the layers of the layered tag providing a unique interrogative signal signature. The tag may be utilized as an identifier.

In another aspect the disclosure relates to a system for treating skin. The system includes a device having a frame and an energy source movably coupled to the frame. A sensor is coupled to the device and is sized and configured to be positioned in communication with a treatment site and is further configured to determine if a topical substance having a desired characteristic is applied to the treatment site. The system includes a mechanism for deactivating the energy emitter if the sensor senses absence of the topical substance at the treatment site. The sensor may be coupled to the energy emitter or may be coupled to the frame. Optionally, a plurality of sensors are coupled to the frame. The sensor(s) may be one or more impedance sensors. The sensor may be configured to distinguish between the topical substance having the desired characteristic and another topical substance not having the desired characteristic. The sensor may be configured to determine if the topical substance having the desired characteristic has expired. In some embodiments, the energy emitter is configured to deliver a desired treatment protocol configured to effect a treatment for a condition such as, for example, acne, unwanted hair, wrinkles, lesions, vascular lesions, or cellulite.

In another aspect the disclosure relates to a skin treatment system including a device configured deliver a therapeutically effective amount of energy to an area of skin and a topical substance configured to be applied to a patient's skin and further configured to have a desired characteristic indicative of an identity of the topical substance. A sensor is coupled to the device and is configured to detect the characteristic of the topical substance. The device includes a mechanism for activating the device only if the sensor detects the characteristic of the topical substance. In some embodiments, the topical substance includes at least one tag configured to exhibit the characteristic of the topical substance. In another embodiment, the topical substance includes a plurality of tags configured to exhibit the characteristic of the topical substance. The topical substance can include at least one tag configured to indicate an expiration date of the topical substance. The mechanism of the system can be configured to de-activate the device if the topical substance is expired based on the expiration date indicated by the tag. In some embodiments, the sensor is configured to distinguish the topical substance applied to skin and the topical substance not applied to skin.

In another aspect the disclosure relates to a topical substance for applying to a tissue surface and including a liquid solution and at least one tag dispersed in the liquid solution. The at least one tag is configured to be identifiable by a sensor.

In another aspect the disclosure relates to a method of initiating tissue treatment, the method includes positioning a sensor in communication with a recognition site of a tissue surface, analyzing the recognition site with the sensor to determine if a desired topical substance is present at the recognition site and activating an energy emitter to deliver energy to a treatment site only if the topical substance is present at the recognition site. In some embodiments, the recognition site is representative of a larger treatment site. In other embodiments, the recognition site is the treatment site. Optionally, the method also includes repeating the activating step so as to treat multiple treatment sites. In some embodiments, the analyzing step is performed prior to each activating step. In other embodiments, the analyzing step is performed only prior to the first analyzing step.

Another aspect of the disclosure relates to a method of initiating tissue treatment including providing a treatment device having an energy emitter at least partially disposed within a handpiece. The device is configured to detect a detectable characteristic of a topical substance applied to a tissue site. The tissue site is analyzed to detect if the topical substance is present at the tissue site. Finally, the energy emitter is activated only if the topical substance is present at the tissue site. The method can also include differentiating between signals from a topical substance disposed over a portion of the tissue and from a bulk volume of the topical substance disposed on the device. For example, the bulk volume of the topical substance is so thick a volume that the presence of the portion of tissue is undeterminable. Alternatively, the bulk volume of the topical substance is a thick “blob” of the topical substance disposed on the device (e.g., the handpiece) and is not in contact with the tissue site.

### BRIEF DESCRIPTION OF THE DRAWINGS

FIG. 1 illustrates an electrode layout suitable for the disclosed impedance sensor. There are 16 small rectangular electrodes on the perimeter of the device footprint outside of the optical window.

FIG. 2 is the block diagram of the control board of the embodiment corresponding to FIG. 1. The electrodes are labeled with capital letters “A” through “P” and correspond to the electrodes shown in FIG. 1 with “A” being located on the top left corner and being placed clockwise around the perimeter of the optical window. The board is connected to the input ports “IMP+” and “IMP−” of the impedance measuring device. Different electrode networks may be created by connecting electrodes with the appropriate switches.

FIG. 3 illustrates the electrode selection logic in an exemplary embodiment of the electrode layout shown in FIG. 1. Each measurement cycle includes the two operation modes. The uniformity mode includes 12 impedance measurements between the separate electrodes over the perimeter of the device footprint. The differentiation mode includes 2 impedance measurements between the opposite sides of the frame while the 4 electrodes on each side are connected together.

FIG. 4a shows the total current density calculated with finite element software where the measurement cycle is in the uniformity mode and where there is tight and uniform contact between adjacent interrogated electrodes.

FIG. 4b shows the total current density calculated with finite element software where the measurement cycle is in the uniformity mode and where there is the presence of small air gap between adjacent interrogated electrodes.

FIG. 5a shows the total current density calculated with finite element software where the measurement cycle has a different implementation of the uniformity mode than is shown in FIG. 4a and where there is tight and uniform contact between adjacent interrogated electrodes.

FIG. 5b shows the total current density calculated with finite element software where the measurement cycle has a different implementation of the uniformity mode than is shown in FIG. 4b and where there is the presence of small air gap between adjacent interrogated electrodes.

FIG. 6 shows the total current density calculated with finite element software for the embodiment shown in FIG. 1 in the differentiation mode where the 4 electrodes present on the opposite sides of the optical window are connected to each other.

FIGS. 7a and 7b show the calibration data and the suggested thresholds for an embodiment of the present disclosure in the differentiation mode as illustrated in FIG. 6. The parameters are: P₁=|Z₃₀|, P₂=Zᵢ₃₀/Z₃₀|, and P₃=|Z₁₀₀/Z₃₀| where Z₃₀ is the impedance at 30 KHz, Zᵢ₃₀ and Zᵢ₁₀₀ are the imaginary parts of the impedance at 30 and 100 kHz, respectively. Different data points correspond to different sensing conditions. The particular sensing conditions are: “JJ”—the device contacts the skin upon which there is a layer of the recommended lotion; “TW”, “SW”, “LO”, “CTP” and “CC”—the device contacts the skin upon which there is a layer of not-recommended (e.g., inadvisable) lotions; and “BU”—where there is bulk unlimited lotion (a layer of lotion measuring at least 5 mm thick on the device), but there is no contact of the device with the skin. The test is designed to show the ability to distinguish the “JJ” lotion from other lotions by sensing the impedance properties of the “JJ” lotion to distinguish it from the other lotions, e.g., the “TW,” “SW,” “LO,” “CTP,” and “CC.” The “JJ” lotion was designed with particular impedance properties that enable it to function as a key that unlocks the device and can begin the cycle that enables the device to fire.

FIGS. 8a and 8b are the same as FIGS. 7a and 7b but show the calibration data and suggest thresholds for an embodiment of the present disclosure in the uniformity mode.

FIG. 9 shows a model of a spherical conductive particle with a shell embedded into an ambient medium.

FIG. 10 is a graph showing the effective dielectric properties of an exemplary multi-phase system vs. frequency (on the logarithmic scale in Hz).

FIG. 11 shows the capacitor geometry where the walls are non-conductive walls (edges).

FIG. 12a is a graph showing the phase angle (a full range of angles) of the impedance for a homogeneous medium, a NaCl solution, (solid line) and multi-phase medium or suspension, NaCl solution in dielectric shells in a dielectric environment,(dashed line) in capacitor geometry vs. frequency.

FIG. 12b is a graph showing the phase angle (the range of angles from −50 degrees to −90 degrees zoomed) of the impedance for a homogeneous medium, a NaCl solution, (solid line) and multi-phase medium or suspension, NaCl solution in dielectric shells in a dielectric environment, real parts (solid line) and the imaginary parts (dashed line) of the impedance of the exemplary multi-phase system in capacitor geometry vs. frequency.

FIG. 13 shows an exemplary layout of a skin sensor model in which there is variable thickness of the topical layer.

FIG. 14a is a graph showing the real part of the impedance of the exemplary multi-phase system vs. frequency for the skin sensor model (logarithmic scale) with no lotion (solid line), lotion at 100 μm (thick dashed line), and lotion at 200 μm (dotted line).

FIG. 14b is a graph showing the imaginary part of the impedance of the exemplary multi-phase system vs. frequency for the skin sensor model (logarithmic scale) with no lotion (solid line), lotion at 100 μm (thick dashed line), and lotion at 200 μm (dotted line).

FIG. 14c is a graph showing the imaginary part of the impedance of the exemplary multi-phase system vs. frequency for the skin sensor model (linear scale) with no lotion (solid line), lotion at 100 μm (thick dashed line), and lotion at 200 μm (dotted line).

### DETAILED DESCRIPTION

The success of professional treatments of a variety of medical, hygienic and cosmetic conditions with directed energy (in particular, light) has excited strong interest in transferring these techniques into the consumer market. However, simple downscaling of the professional technology is not an option for such technology transfer. Attaining acceptable levels of efficacy and safety in a self-use device requires complex technical solutions. A professional-grade treatment in a consumer device is a desired endpoint.

In one embodiment, a compound (a topical or a lotion) and apparatus (sensor) are combined to ensure safe delivery of the electromagnetic energy only to areas of the human body designated for treatment. Suitable sensor and lotion systems include one or more of: (1) a reduced and/or minimized probability of energy emission on undesired areas of human body (such as an open eye or a closed eye), (2) determining that the lotion is present prior to treatment of an area of tissue in order to maximize efficacy of treatment, and (3) enhanced ergonomics that facilitate use of the device.

Impedance Sensor System

Light treatment of skin conditions such as wrinkles, pigmented spots, undesirable hairs, port-wine stains, and other vascular disorders, can only be efficient if the light intensity is sufficiently high. On the other hand, the use of the high-intensity light from light sources such as lasers or lamps generally require appropriate safety measures to be undertaken for avoiding skin and/or eye injury. Safety measures may include both instructing the personnel that use the high intensity light devices and using engineering controls. Use of engineering controls is important for use by less educated providers (e.g., salon employees, aestheticians) and for home-use devices operated by consumers (e.g., untrained non-professionals).

As discussed herein, combining high spatial resolution with the low probability of sensor failure is a challenging task. Fulfilling that challenge can enable the engineering controls necessary for skin and/or eye safety with high intensity light devices. Impedance sensor(s) can fulfill the requirements of this task as they show high spatial resolution. Impedance sensors can be used, in particular, for providing both skin safety and eye safety when treating different skin conditions with high-intensity light.

A typical approach for providing skin safety is contact cooling of skin. For example, a chilled optical window is kept in tight contact with skin during the light pulse and, if necessary, before and after the pulse as well. An important part of the contact cooling approach is the use of a lotion specially designed for the particular treatment type. The lotion should show a high thermal conductivity for providing good thermal contact of skin with the optical window and a proper optical refractive index for coupling the treatment light into the skin.

The general problem with contact cooling is that firing may only be allowed if contact is tight and uniform over the whole optical window and the right lotion is put on the treatment site; otherwise, a serious thermal injury can occur. Practically, it is often difficult to keep track of all the safety conditions during the treatment procedure. For instance, the non-professional operator can loosen contact or go out of the lotion zone rather easily. The conventional technical solutions comprising the use of mechanical and electrical contact sensors might not detect the unsafe situations reliably. The mechanical sensors of certain types can check the presence of contact, the contact uniformity, and the applied pressure, but fail with the lotion evaluation. The spatial resolution of the mechanical sensors can be limited by the pin separation because they do not cover the area between the pins. Some known bioelectrical sensors are designed for taking the impedance measurements of the whole body or of certain internal organs. Such approaches mostly use the 4-electrode layout, big electrode size, and cover large skin areas (much bigger than typical footprint size of a light based device that is in the range of about 1-5 square centimeters). Simply scaling the same layout down to a small size can result in low signal levels and compromises the detection reliability thereof.

The development of the engineering controls for eye protection during light treatment of skin is desirable. Eye detection technologies are especially important for in-home treatment of the periorbital skin area. Some previous approaches use either the “red eye” effect caused by the reflection of light from the eye retina, the specular reflection of the outer eye surface (often combined with the optical flow detection), or the morphological segmentation of face pictures. All these optical approaches fail in the potentially unsafe situation where a small part of the eye is only present within the treatment zone.

In accordance with one aspect of the disclosure an impedance sensor has a set of relatively small electrodes located on the perimeter of the device footprint around the optical window. The electrodes may be gold-plated or covered with a different electrically conductive layer preventing electrolysis and electrode decay when contacting skin. Each electrode can be supplied with a separate wire connecting the electrode to control board with a set of switches. The output pins of the control board are connected, in turn, to impedance measuring device (IMP). The switches are controlled by a certain program and can flip at a certain rate changing the configuration of the electrode networks. At each time instant several electrodes can be connected to the IMP while the others are disconnected. The IMP device can work in either AC or DC mode.

In another aspect of the disclosure the measurement procedure can include several time steps using different electrode networks on each step. In the AC mode the complex impedance or admittance values may be measured at several carrier frequencies. The impedance or admittance values measured hereby can depend on the contact conditions between the device footprint and skin. After all the predefined time steps are done, a certain decision algorithm can be applied to determine the firing status. The procedure of determining the firing status can be repeated periodically.

In still another aspect of the disclosure, an algorithm can be used for determining the firing status given by the impedance or admittance values measured previously. In one embodiment the algorithm makes just a binary choice between the “allowed” and “prohibited” status options. In other embodiments additional status options may be considered, for instance, “stop procedure” or “delay firing.” Preferably, the algorithm includes the two steps: (a) evaluation of several parameters as functions of the measured impedance values; and, (b) comparison of the parameter values to certain thresholds. The outcome of the algorithm is the firing status, e.g., “allowed” or “prohibited.”

In yet another aspect of the disclosure the appropriate calibration procedure is used for the evaluation of the aforementioned thresholds for the parameter values. Preferably, the calibration procedure uses the logging mode of the sensor electronics. In this mode the determination of the firing status is “off” and the operator can download the measured data from the internal memory of the impedance measuring device to a computer. The operator recruits several subjects and makes impedance measurements with each subject for a predefined list of contact conditions, for instance:
1. Footprint is in tight contact with skin and the recommended lotion is applied.
2. Footprint is in tight contact with skin and an inadvisable (e.g., not recommended) lotion is applied.
3. Footprint is in tight contact with skin and no lotion is applied.
   Next to this, the full set of contact conditions to be examined may include those with no subject, for instance:
4. No skin contact and no lotion on footprint.
5. No skin contact and a thick layer of the recommended lotion on footprint.
   After all the calibration measurements are done, the operator can download the measured data to the computer and use certain software for the threshold determination. Given the threshold data, the sensor electronics can be reprogrammed and tested for the correct operation.

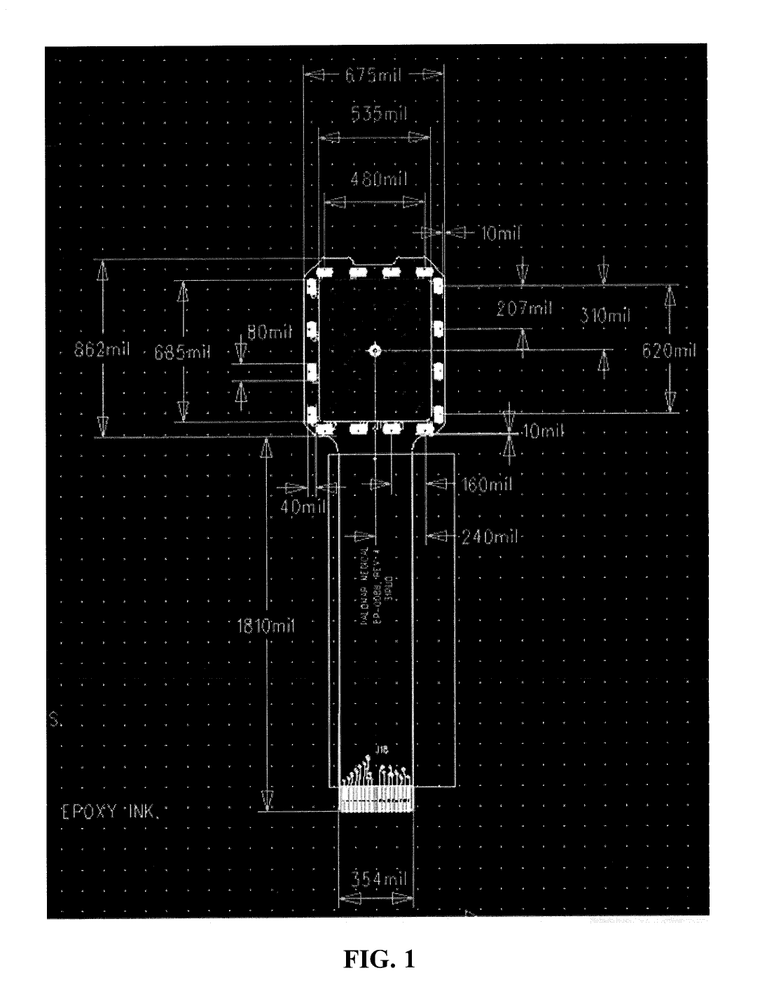

The electrode layout shown in FIG. 1 illustrates a sensor layout with 16 rectangular electrodes the electrode layout is suitable for the disclosed impedance sensor. There are 16 small rectangular electrodes on the device footprint (e.g., about the perimeter of the device footprint and/or around the optical window of the device in a substantially rectangular pattern and/or attached to the device frame). The 16 small electrodes are referred to by capital letters “A” through “P.” The electrode A is located in the top left corner and the sixteen electrodes are in the clockwise direction starting from the top left corner A through P. The electrodes may be made from any suitable material. For example, the electrodes may be made of nickel and/or copper and/or gold plate. In one embodiment, the distance between the centers of adjacent electrodes (e.g., the distance between the center of electrode A and the center of electrode B or the distance between the center of electrode A and the center of electrode P) should be from about 1 mm to about 5 mm, or from about 2 mm to about 4 mm. The distance between adjacent electrodes defines the dimensions of the smallest air gap that can be detected; more particularly the closer packed the electrodes (e.g., the shorter the distance between adjacent electrodes) the more sensitive the detector is to small gaps in lotion coverage. The device sensor can be tuned to distinguish between determining normal variation in skin topology (e.g., pock marks, scars, dimples, etc.) and determining the presence of a corner of an eye, which is a goal of differentiation using the sensor. FIG. 1 illustrates 16 electrodes around a substantially rectangular perimeter, but the number of electrodes, the shape of each electrode and the shape of the perimeter surrounded by the electrodes may vary. For example, the number of electrodes can be suited to the desired level of sensitivity, the size of the treatment area, and the shape of the footprint (e.g., the footprint could have an amorphous shape, be round, or have another shape). The number of electrodes that are about the perimeter of the device is at least 2 and could be up to thousands of electrodes depending on the desired contact detection level, the size of the treatment area, etc.

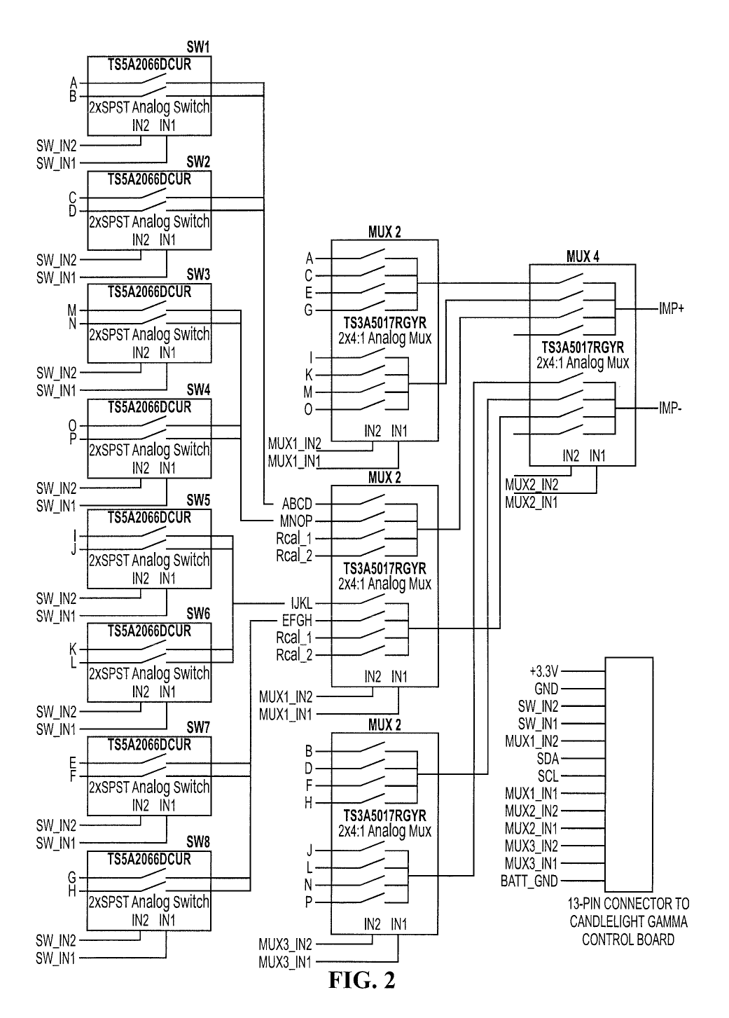

FIG. 2 shows a block diagram of the control board for the sensor layout shown in FIG. 1. The electrodes are connected to the control board with switches and input pins, IMP+ and IMP− of the impedance measuring device. The electrodes in the block diagram are labeled with capital letters A through P that correspond to the electrodes shown and described in FIG. 1. The board is connected to the input ports “IMP+” and “IMP−” of the impedance measuring device. Different electrode networks may be created by connecting electrodes with the appropriate switches.

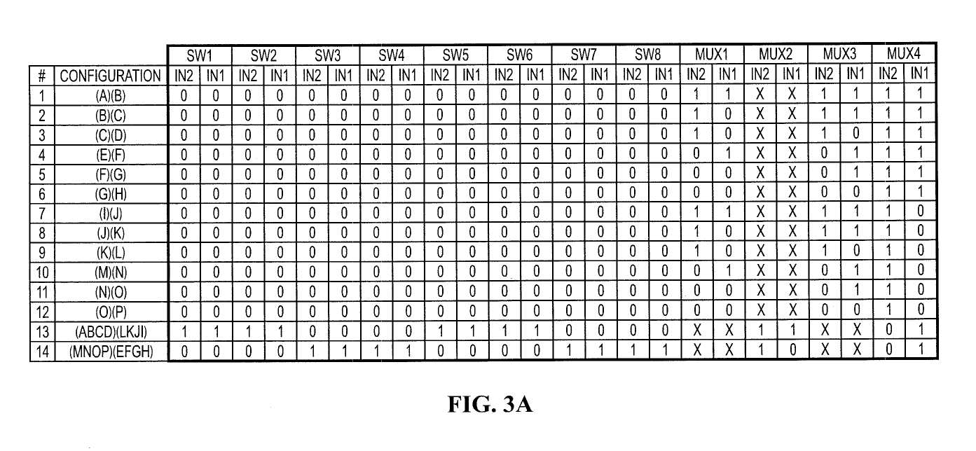

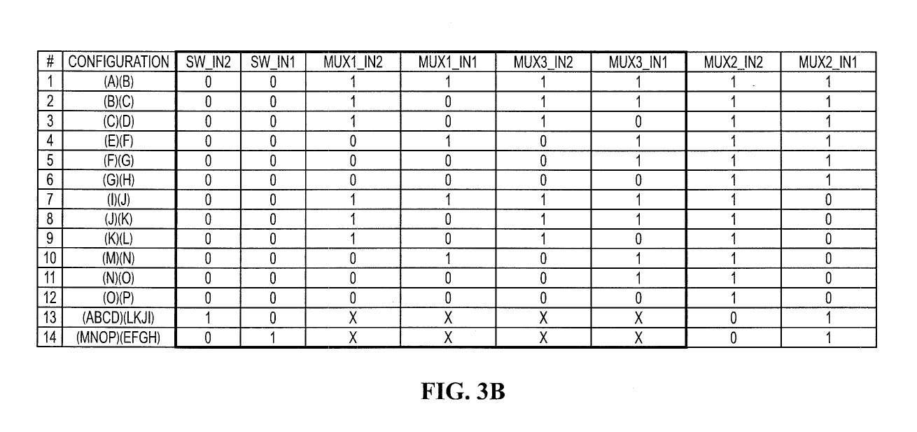

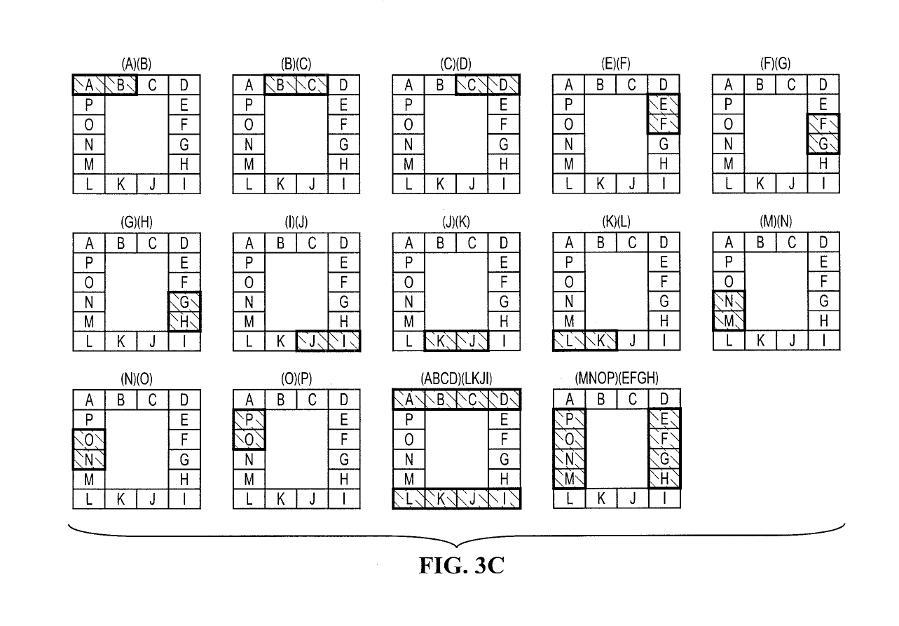

FIG. 3 illustrates the impedance electrode selection logic for the sensor shown in FIG. 1 and for the control board shown in FIG. 2. FIG. 2 also shows a series of 14 configurations that the rectangular electrodes go through. Each measurement cycle includes two operation modes, namely the uniformity mode and the differentiation mode. The uniformity mode includes 12 impedance measurements (in FIG. 3 configuration #1-#12) between the separate electrodes around the perimeter. The uniformity mode evaluates the uniformity of contact of the electrodes about the perimeter of the optical window. The uniformity mode includes the 12 first impedance measurements, namely configuration #1 (A)(B) through configuration #12 (O)(P) shown in FIG. 3. In the uniformity mode each measurement is between the 2 electrodes while keeping all the other electrodes disconnected.

Evaluating the differentiation mode includes 2 impedance measurements between the opposite sides of the frame while the 4 electrodes on each side are connected together (in FIG. 3 configurations 13 and 14). It is not required for the differentiation mode that the electrodes be directly across from one another as is illustrated in FIG. 3, rather the electrodes must be in pairs for the differentiation mode to interrogate the electrodes.

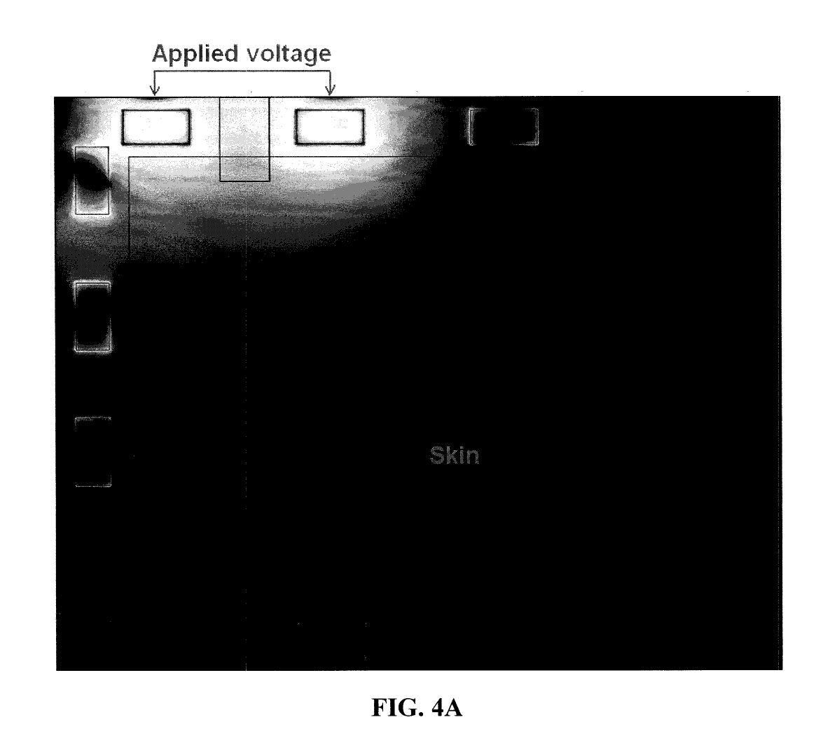

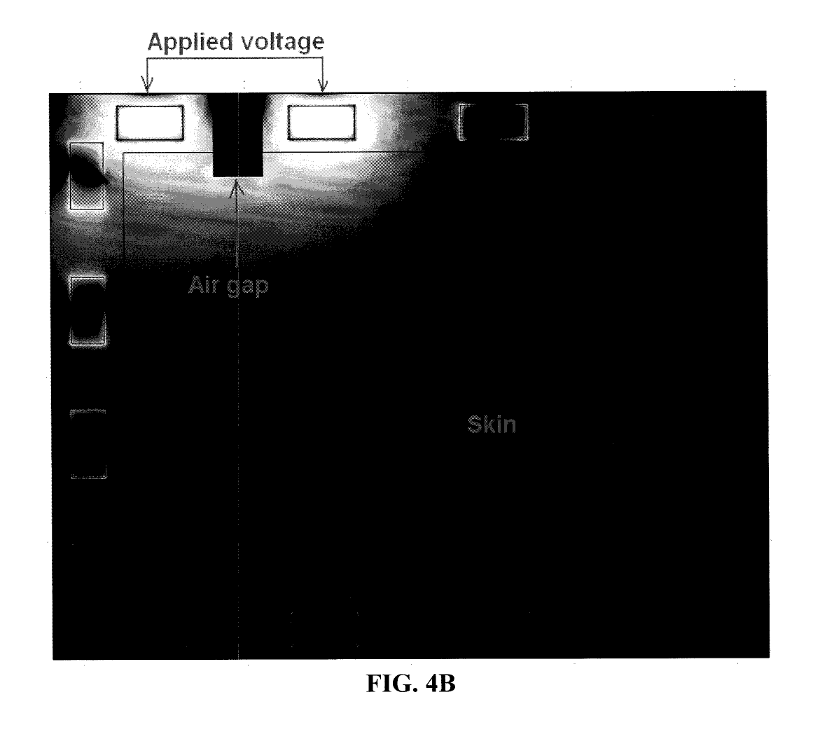

Measurement in the uniformity mode (specifically, in configuration #1 shown in FIG. 3) is illustrated in FIGS. 4a and 4b, which provide a calculated profile (calculated with finite element software Comsol 3.5a) of the total current density for the 16-electrode sensor positioned about the perimeter of the optical window. FIG. 4a shows the calculated total current density profile using the logarithmic current density scale for the 16-electrode sensor contacting wet human skin in the uniformity mode in the case of tight uniform contact between the sensor and the wet human skin. As discussed here, wet human skin means human skin that is substantially uniformly covered with lotion.

FIG. 4b shows the calculated total current density profile (specifically, in configuration #1 shown in FIG. 3) using the logarithmic current density scale for the 16-electrode sensor contacting wet human skin in the uniformity mode in the case of the presence of an air gap (e.g., a relatively small air gap that provides a break in the presence of otherwise uniform coverage of lotion on human skin). The air gap can illustrate a region of the human body such as the corner of a human eye where lotion would not be present (e.g., during a treatment for lines adjacent to a human eye commonly called crow's feet).

FIG. 4a shows the calculated plot of the total current density in the case of tight and uniform contact between the electrodes about the perimeter of the optical window and the skin while FIG. 4b shows the similar plot in the presence of a small air gap on skin between the electrodes A and B about the perimeter of the optical window and the skin. Comparing FIGS. 4a and 4b shows that the air gap (e.g., in FIG. 4b) increases the path length between the electrodes and the impedance thereof. FIGS. 4a and 4b show one embodiment of the technique for detecting an air gap in the presence of lotion coverage. This technique employs the absolute value of the current to determine the presence of an air gap. The presence of the air gap can be revealed by a relatively lower current than where there is the absence of an air gap. The lighter gray area in FIGS. 4a and 4b around the region of Applied Voltage illustrates how the current density is impacted by the air gap. FIGS. 4a and 4b show that about the axis of symmetry 50 there is a substantially symmetrical current density both in the presence and in the absence of an air gap.

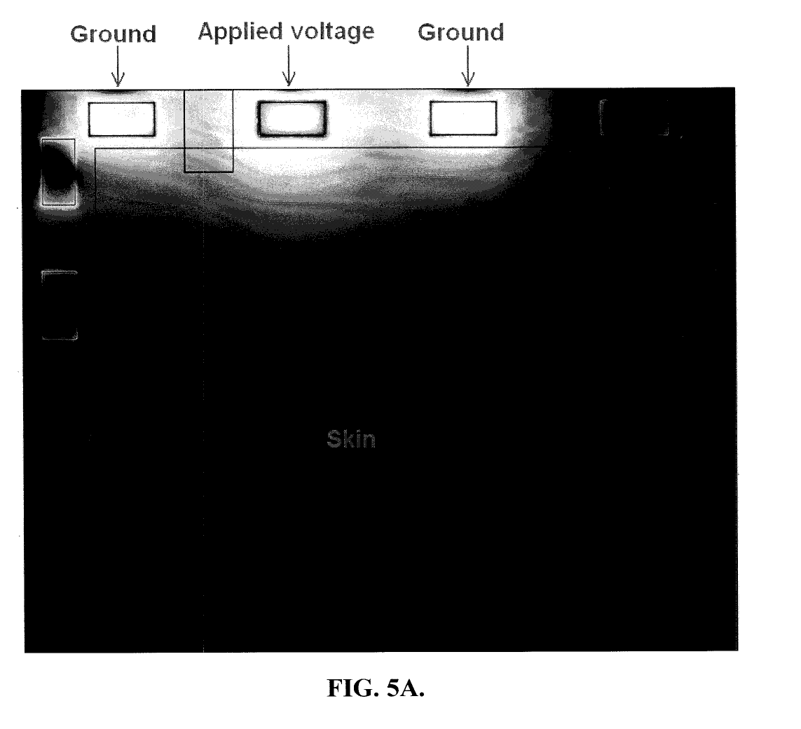

FIGS. 5a and 5b show a different implementation of the uniformity mode illustrated in FIGS. 4a and 4b. Specifically, FIGS. 5a and 5b differ from what is shown in FIGS. 4a and 4b in that the signal is applied to a certain electrode while the 2 surrounding electrodes are connected to the common ground. FIGS. 5a and 5b show one embodiment of the technique for detecting an air gap in the presence of lotion coverage; this technique employs the use of the difference of the measured current values between two neighboring pairs of electrodes to determine the presence of an air gap in the lotion. Here the presence of a gap is revealed by a lower current than in the absence of a gap in the lotion.

The technique described in association with FIGS. 5a and 5b is more robust than the technique described in association with FIGS. 4a and 4b, because it is more resistant to the impact of environmental noise on the level of the current then the technique shown in FIGS. 4a and 4b. The lighter gray area in FIGS. 5a and 5b around the region of Applied Voltage illustrate how the current density is impacted by the air gap. FIGS. 5a and 5b show that about the axis of symmetry 50′ there is a difference in the current density in the presence of the air gap that enables detection of the air gap. This is because rather than measuring the absolute value of the current, instead the difference between the current between two neighboring pairs of electrodes is measured (e.g., the subtraction method) which is more robust. More particularly, because the integral of the current density in the region between the outer perimeter of the current density region and the axis of symmetry in the presence of an air gap has a lower value than the integral of the total current between the outer perimeter of the current density region and the axis of symmetry in the absence of an air gap. In the alternative implementation of the uniformity mode shown in FIGS. 5a and 5b, one measures the impedance difference between a central (signal) electrode (e.g., electrode B) and the 2 side (ground) electrodes (e.g., electrode A and electrode C). The subtraction method may reduce noise due to the unstable applied pressure, different conditions on skin surface, uneven skin thickness, etc. as a result sensitivity to the localized breaks of contact, for instance, air gaps and lotion bubbles is improved.

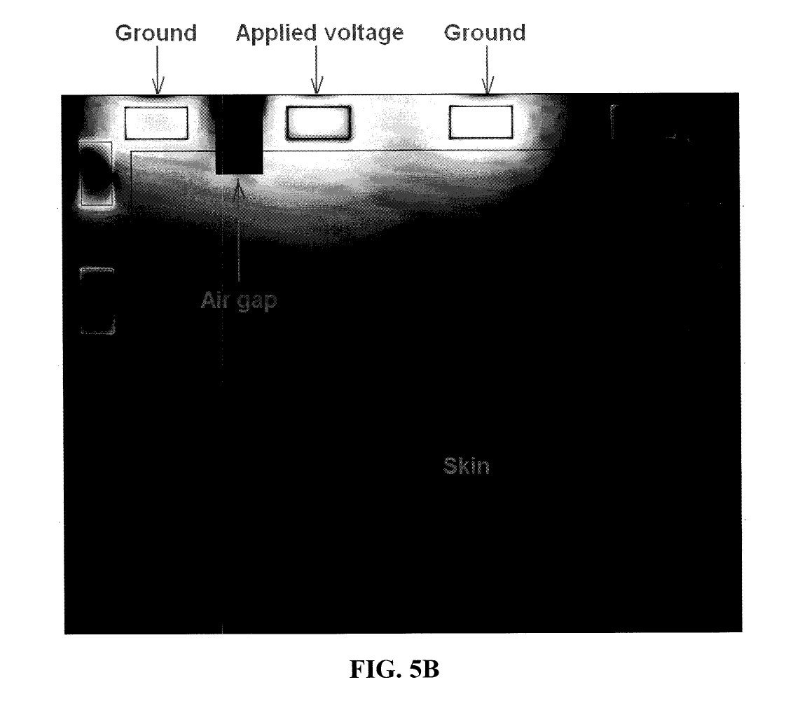

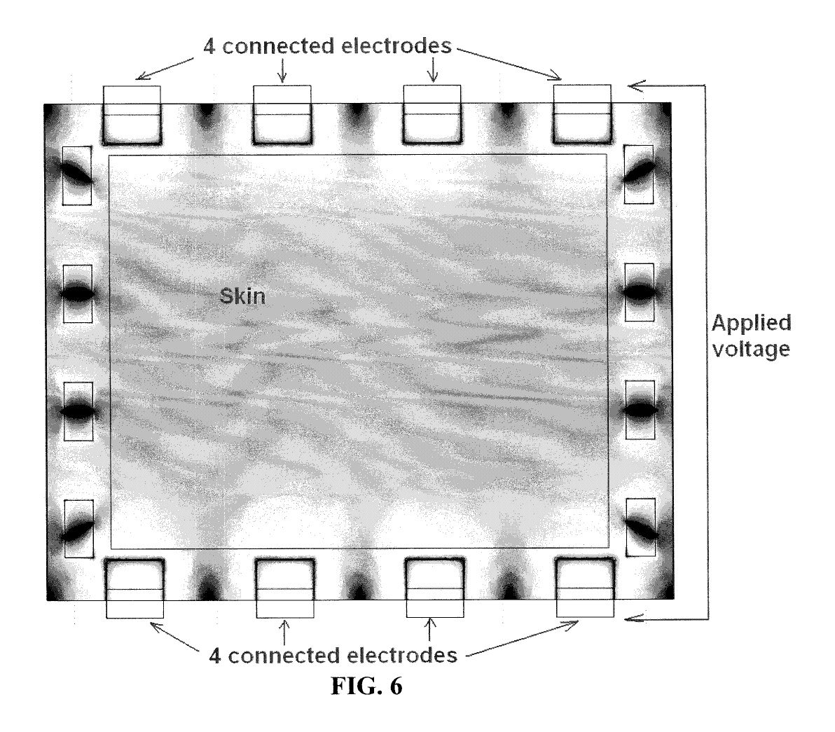

FIG. 6 shows a calculated profile using the logarithmic current density scale (calculated via finite element software Comsol 3.5a) of the total current density for the 16-electrode sensor contacting the wet human skin (e.g., human skin that is substantially uniformly covered with lotion) in the differentiation mode where the four electrodes on one side and the four electrodes on the opposite side of the optical window are connected to one another. The differentiation mode (or topical differentiation mode) includes the 2 impedance measurements shown in FIG. 3 that correspond to Configurations #13 and 14. Each measurement is between the 2 opposite sides of the optical window where the electrodes are connected while the other eight electrodes (e.g., two sets of four electrodes on the other two sides of the perimeter) are disconnected. FIG. 6 illustrates the differentiation mode Configuration #13 as shown in FIG. 3.

The two operation modes (e.g., the uniformity mode and the differentiation mode) provide a benefit by being combined. The use of small electrodes about the perimeter of the window in the uniformity mode provides good spatial resolution over the perimeter of the window at the expense of low impedance resolution, because only two electrodes are interrogated at a time. A break of contact can be detected in the uniformity mode even if being localized in a small area. However, a limitation of some embodiments of the uniformity mode is that different topicals on the skin surface cannot reliably be differentiated. However, in the differentiation mode the multiplexors on the control board are used to connect several electrodes together thereby increasing the signal to noise ratio and making the topical differentiation possible. The downside of the differentiation mode is the drop of spatial resolution. Combining the two complementary operation modes in the same measuring cycle can provide high spatial resolution and high impedance resolution in a single measuring cycle.

The algorithm of an embodiment of the impedance sensor may be outlined as follows. Every uniformity mode measurement and every differentiation mode measurement yields two complex impedance values (e.g., Z₃₀ and Z₁₀₀) determined at carrier frequencies (e.g., 30 and 100 kHz), respectively. Thus, in each mode four numbers (e.g., Zᵢ₃₀, Z₃₀, Zᵢ₁₀₀, and Z₁₀₀) that constitute two complex numbers (e.g., Z₃₀ and Z₁₀₀) are measured that enable determination of the contact status of the sensor and what action the sensor may take (e.g., enable firing or disable firing etc.). Where subscript where subscript i stands for the imaginary part of impedance. The software (e.g., software in the system) utilizes these two complex numbers to calculate the three real parameters (e.g., P₁, P₂ and P₃) characterizing the contact status:

P₁=|Z₃₀|, P₂=|Zᵢ₃₀/Z₃₀|, and P₃=|Z₁₀₀/Z₃₀|.

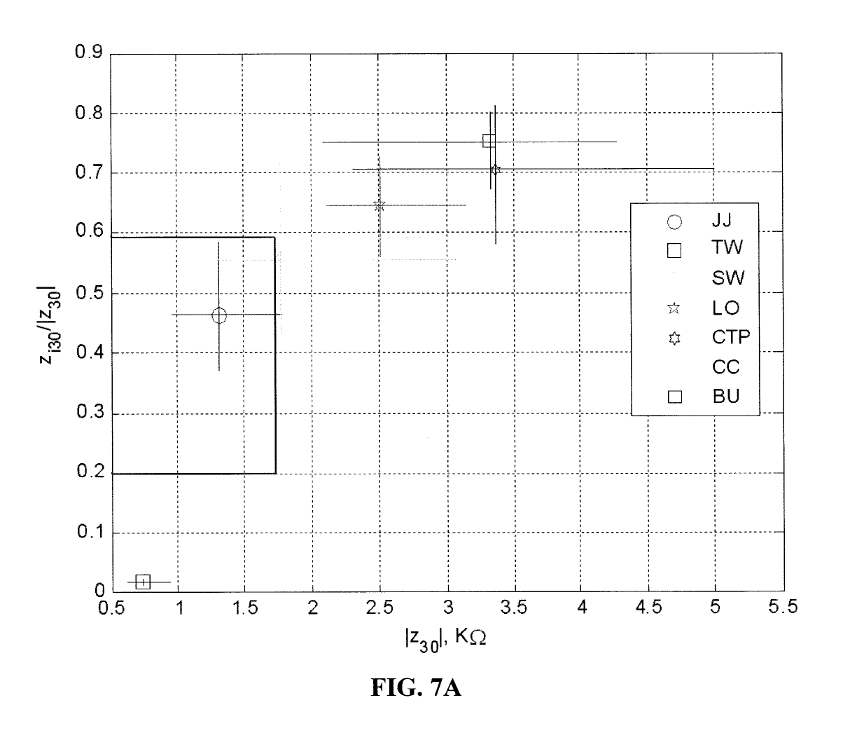

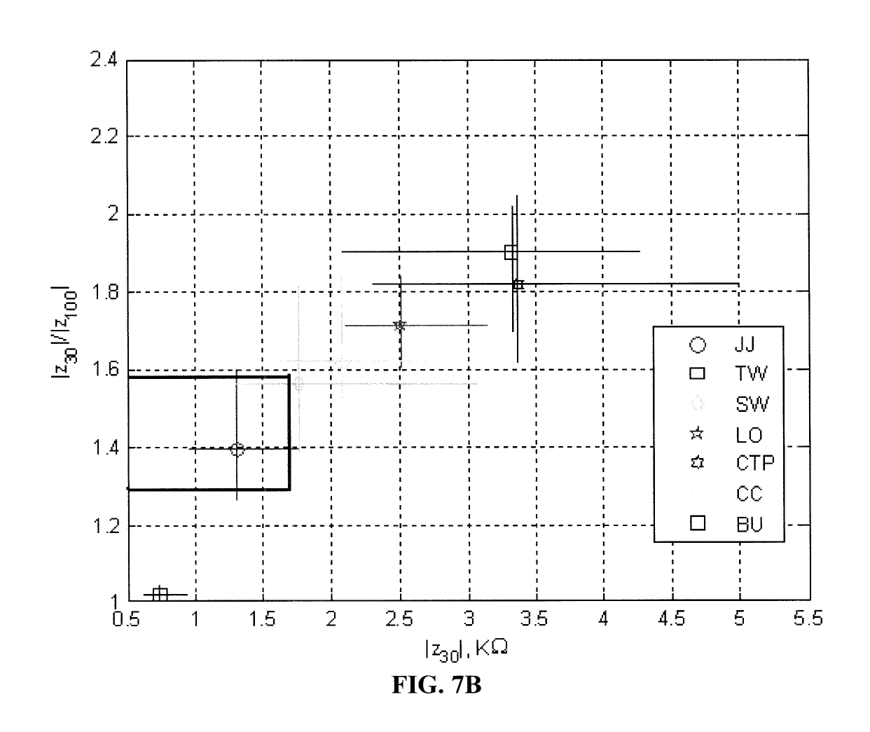

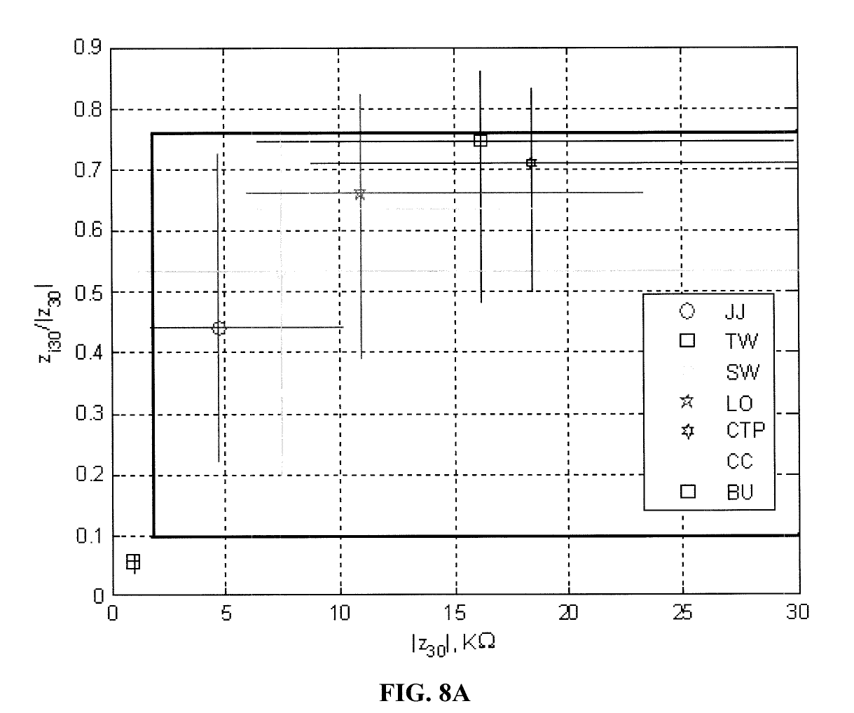

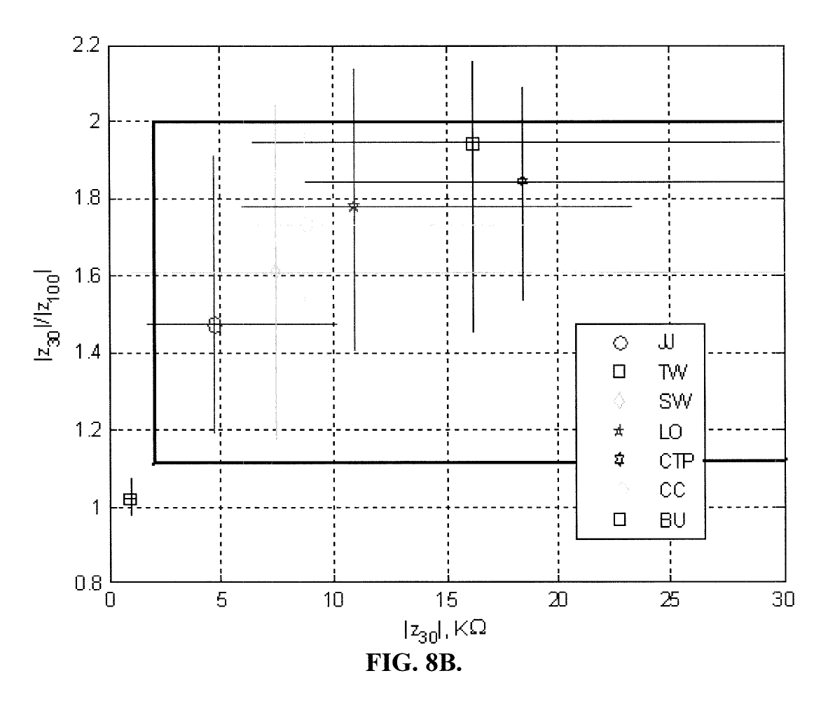

The firing can be allowed only if all three of the parameters are within certain limits as illustrated in FIGS. 7a and 7b for the differentiation mode and FIGS. 8a and 8b for the uniformity mode. The complex impedance values Z₃₀ and Z₁₀₀ are only exemplary. In another embodiment, different complex impedance values could be measured at different frequencies to provide similar simplified detection criteria in the form of, for example, one or more real parameters that characterize the contact status.

More specifically, FIGS. 7a and 7b show the calibration data and the suggested parameter thresholds for an embodiment of the present disclosure in the differentiation mode discussed in association with FIG. 6. The particular conditions to be checked in this case in the differentiation mode are as follows:
1. P₁<1750Ω,
2. P₂<0.59,
3. P₂>0.2,
4. P₃<1.59,
5. P₃>1.25.

FIGS. 8a and 8b show calibration data and the parameter thresholds for the uniformity mode of the exemplary sensor. The particular conditions to be checked in this case in the uniformity mode are as follows:
1. P₁>2000Ω,
2. P₂<0.75,
3. P₂>0.1,
4. P₃<2,
5. P₃>1.1.

The firing status is set to “allowed” only if the five differentiation mode conditions and the five uniformity mode conditions above are “true,” otherwise, the firing status is set to “prohibited.”

In this embodiment, the threshold values for all the three parameters namely, P₁=|Z₃₀|, P₂=|Zᵢ₃₀/Z₃₀|, and P₃=Z₁₀₀/Z₃₀|, for both the operation modes (e.g., the uniformity mode and the differentiation mode) may be evaluated using a calibration procedure. Calibration measurements are performed with several subjects for the safe firing conditions using a recommended lotion. The additional measurements may be performed for inadvisable topicals and other unsafe firing conditions. The thresholds must be chosen to include most the data points obtained under the safe firing conditions (e.g., JJ) and reject those obtained under the unsafe conditions. FIGS. 7a, 7b, 8a and 8b show the diagrams of the calibration data for the embodiment of the impedance sensor discussed in association with FIGS. 1-6 and calculated via Matlab R2008a, The MathWorks, Inc., 3 Apple Hill Drive, Natick, Mass. 01760-2098, United States for the recommended topical on skin (JJ), several inadvisable topicals (e.g., not-recommended topicals) on skin (TW, SW, LO, CTP and CC), and no skin contact, but instead with a thick layer of the recommended topical on the device footprint (BU). The recommended lotion JJ can include DI water, Glycerine, Ultrez 10, Sodium Hydroxide (20% stock solution) (0.1% by weight), Sodium Chloride (0.1% by weight) and a preservative. The pH of the recommended lotion JJ is adjusted to be in the range of from about 6.4 to about 6.6. The thickness of the lotion as applied to the skin should range between about 10 microns and about 500 microns. The error bars show the full span of the data points. This example of the differentiation mode shown in FIGS. 7a and 7b demonstrates the possibility of differentiating skin surface conditions by applying the appropriate thresholds via use of a recommended topical on the skin, for example, the topical labeled JJ.

More specifically, FIGS. 7a and 7b show the calibration data and the suggested parameter thresholds for an embodiment of the present disclosure in the differentiation mode discussed in association with FIG. 6. The parameters are: P₁=Z₃₀|, P₂=|Zᵢ₃₀/Z₃₀|, and P₃=|Z₁₀₀/Z₃₀| where Z₃₀ is the impedance at 30 kHz, Zᵢ₃₀ and Zᵢ₁₀₀ are the imaginary parts of the impedance at 30 and 100 kHz, respectively. Different data points on FIG. 7a correspond to calibration data and FIG. 7b show the parameter thresholds for the differentiation mode of the exemplary sensor in different sensing conditions. The particular sensing conditions are: JJ contact with skin with a recommended lotion where the recommended lotion is applied in a thickness range of from about 10 microns to about 500 microns. The sensing conditions TW, SW, LO, CTP and CC contact with skin with different inadvisable (non-recommended) lotions applied in a thickness range of from about 10 microns to about 500 microns. The sensing condition BU shows where there is bulk unlimited lotion (a layer of lotion measuring at least 5 mm thick), but no contact with skin.

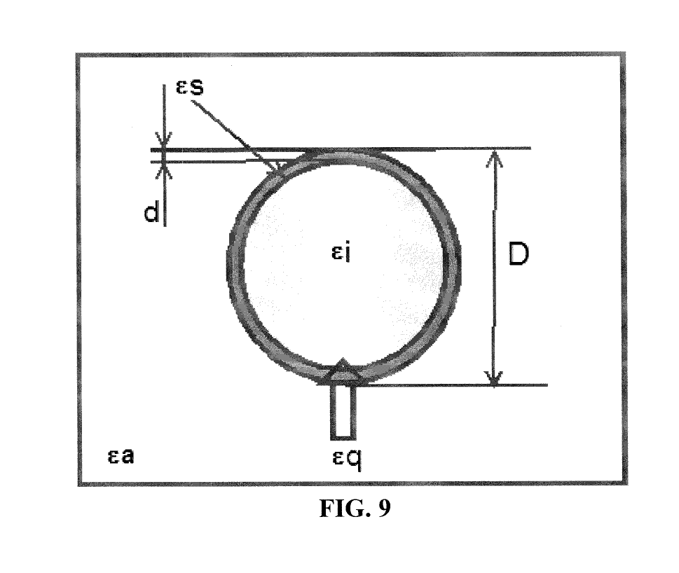

FIGS. 7a and 7b provide a test designed to show the ability to distinguish the recommended JJ lotion from the other inadvisable lotions TW, SW, LO, CTP and CC lotions. The JJ lotion was designed with particular properties to enable it to be distinguished from the other inadvisable lotions TW, SW, LO, CTP and CC. The JJ lotion was designed to function in the lock and key fashion and the test depicted in FIGS. 7a and 7b show that the recommended lotion JJ can be distinguished from the other lotions via sensing the impedance values of the lotions in the test. Here the enhanced impedance properties of the target lotion JJ enabled the target lotion JJ to be differentiated from other cosmetic products. Also in order to be able to differentiate the JJ lotion it needed to be tailored to be different from other lotions on the market/in the test such that it displayed impedance values that are capable of being differentiated from the other lotions. Here the lotion impedance value was able to be differentiated because of an increase in the salt content of the JJ lotion relative to the other inadvisable lotions, TW, SW, LO, CTP and CC . This strategy of increasing the salt content to make the lotion able to be differentiated must be used with caution, because there are risks of false differentiation because there could be other salty mediums in or on the skin that could trick the sensor and enable the sensor to signal that firing the device should be enabled under unwanted conditions. The risks of tricking the sensor due to salt content on the skin and high salt content lotions on the market is why a multiphase topical (described below in accordance with FIGS. 9-14c), which is more complex, might be desirable, because it is more robust and will make the lotion truly unique.

Referring still to FIGS. 7a and 7b, the firing zone 100 is the zone outlined by a rectangle surrounding the lotion JJ and is the zone where firing is allowed. The part of the parameter space exterior to the firing zone 100 is the zone 120 where the firing is prohibited.

The zone where firing is allowed is outlined by a rectangle is the “firing zone” 100 and in FIGS. 7a and 7b this is the region showing that lotion “JJ” is sensed. The part of the parameter space exterior to the rectangular firing zone 100 is the zone 120 where the firing is prohibited.

FIGS. 8a and 8b are the same as FIGS. 7a and 7b, but the calibration data and the parameter thresholds are for the uniformity mode of the sensor. In FIGS. 8a and 8b, the “firing zone” 100 covers a larger range of lotions than in FIGS. 7a and 7b. The criteria that are required to be met to allow firing in the “firing zone” 100 is the presence of lotion without air gaps are relaxed compared to the criteria in FIGS. 7a and 7b that ensure that the recommended lotion is present and firing is allowed only in the presence of the recommended lotion.

In some embodiments, an impedance sensor is employed for a light-based device for skin treatment. The impedance sensor determines the proper conditions on the skin surface for firing. In one embodiment, the sensor includes a set of electrodes around the optical window of the light-based device, a control board with switches, and an impedance measuring unit. The switches can connect the electrodes together or disconnect them forming different electrode networks.

In some embodiments, the sensor may perform a predefined set of operations periodically in time. The predefined set of operations may include several impedance measurements using different electrode networks and processing of the measured data with a certain algorithm for the determination of the firing status. In some embodiments, the impedance measurements include two modes: a mode for the topical differentiation characterized by a high susceptibility to the impedance value (FIGS. 7a and 7b) and a mode for the evaluation of the contact uniformity characterized by a high spatial resolution (FIGS. 8a and 8b). These modes will be referred to as the differentiation and uniformity modes, respectively. Preferably, in the uniformity mode (e.g., FIGS. 8a and 8b) the electrodes may be interrogated sequentially by pairs or triples while the other electrodes are disconnected. If the electrodes are interrogated in triples the central electrode may be the signal one while the side electrodes may be connected to the same ground. The impedance values are measured between the signal electrode and each of the ground electrodes. The difference between the impedance values is not susceptible to large-scale impedance variations but may still be susceptible to localized air gaps and lotion bulbs. Preferably, in the differentiation mode (e.g., FIGS. 7a and 7b) the impedance values are measured between the connected electrodes on the opposite sides of the optical window.

In some embodiments, the algorithm computes several functions of the measured data and compares the calculated values and the predefined thresholds. The firing may be allowed only if all the checks have been passed. For each sensor layout, a certain calibration procedure must be performed for the determination of the predefined thresholds.

Thus, in an exemplary implementation, the body treatment device is powered and the optical window is stamped on the surface of a skin tissue upon which a topical has been applied. There are a number of electrodes on the perimeter of the device footprint outside of the optical window. The device is initially run in differentiation mode (see, e.g., FIG. 3 configurations 13 and 14 and FIGS. 7a and 7b), which enables the device to differentiate if the “correct” topical (e.g., JJ) is located on the skin. If the correct topical is determined in the differentiation mode then the uniformity mode (see, e.g., FIG. 3 configurations 1-12 and FIGS. 8a and 8b) is employed to determine if the device is evenly (e.g., substantially uniformly) positioned on the subject's skin and that air gaps are not present in the lotion. When it is determined that the device is uniformly positioned on the subject's skin then then emission of a first pulse of the scan is enabled. After the first pulse, the uniformity mode (see, e.g., FIG. 3 configurations 1-12) is employed again to determine if the device continues to be evenly (e.g., substantially uniformly) positioned on the subject's skin. When it is determined that the device remains uniformly positioned on the subject's skin, then the emission of the next pulse of the scan (e.g., the second pulse) is enabled. Evaluation of the skin area via the uniformity mode is repeated until every pulse in the scan is complete. If the uniformity mode indicates an interruption, such that the device is not in the desired even position on the skin or such that there is an air gap present in the lotion disposed on the skin then the test fails and the scan is interrupted. The device can respond to the interruption in any of a number of ways, for example, the uniformity mode can be repeated after the interruption or a new scan can be required. In one embodiment, in a treatment using a stamping technique to accomplish treatment, the differentiation mode is repeated after each stamp at the beginning of each new scan to ensure that the device is functional only upon contact with a desired topical (e.g., JJ).

This concept that ensures the recommended lotion is employed and that the treatment device has uniform contact could extend to treating a subject in the continuous wave (CW) mode. The specific numerical criteria (e.g., the parameters for the firing zone, etc.) could be adjusted to accomplish this objective. In accordance with a CW mode of treatment, one would likely continue to have a differentiation mode and a uniformity mode.

Multi-Phase Conductive/Dielectric System

In one embodiment, the system and/or the device is able to identify a topical (such as a lotion) that contains one or more markers. Such a device and lotion system could be used with any directed energy device application. For example, in a professional setting lesser educated professional such as a salon employee may use such a topical/lotion and sensor system. In another embodiment, such a topical/lotion and sensor system can be for home use.

In order to implement the use of lotion having markers in the compound, multi-phase systems may be employed. In a multi-phase system, two or more phases (e.g., background solution vs. suspended particles) provide additional means for increasing contrast of the lotion signal vs. no-lotion signal. In one embodiment, contrast is increased many fold by employing two or more active components in the background solution, in the suspended particles, and/or at least one active component in each of the background solution and in the suspended particle(s). Alternatively or in addition to exploiting the physical properties of the active component(s), the non-uniform distribution of the active components in the solution can enhance the contrast and improve differentiation from other topicals/lotions that might be present on the skin.

In one embodiment of a multi-phase system, electromagnetic means are employed in which particle spheres float in a solution. In one embodiment, in a dielectric solution, particles with electromagnetic properties substantially different from the surrounding background are suspended in the dielectric solution. For example, conductive spheres can be suspended in a dielectric solution. This will result in impedance spectra of such a multi-phase solution being substantially different from the impedance spectra of a homogenous solution (e.g., a dielectric solution without suspended spheres). In another embodiment, non-conductive spheres are suspended in a conductive solution, which will result in impedance spectra of such a multi-phase solution being substantially different from the impedance spectra of a homogenous solution (e.g., a conductive solution without non-conductive suspended spheres).

For example, in one embodiment, a multi-phase system includes a composition of 20% (V:V) Barium Ferrite (Sigma-Aldrich #383295-250G) dispersed in a highly conductive medium (180 g PEG dissolved in 400 mL deionized water, add 30 g NaCl (also dissolved) and mixed with 150 mL of glycerin). This Barium Ferrite containing multi-phase system was tested on a non-conductive surface (glass plate) and on a conductive surface (forearms of experimenter). The composition containing Barium Ferrite containing multi-phase system showed significant increasing of phase and high resistance of composition. It is desirable that the topical and/or the dispersed composition be translucent (or substantially translucent). For example, in one embodiment, the translucence requirement for a dispersed composition, the topical and/or the lotion is that there is an absorption coefficient at the wavelength of interest that does not exceed about 20 mm⁻¹ (so called inverse mm), which is a measure of translucency. At least substantial translucency is desired so that light can travel through the composition. It is also desirable that the dispersed composition has magnetic permeability. It is desirable that the compound within the particles encapsulated in the topical have magnetic permeability, because when an external magnetic field is applied the domains within the compound may be oriented in a desirable manner. Higher magnetic permeability could allow achievement of a positive phase. Thus, in the embodiment of a composition containing a Ferrite such as Barium Ferrite it is desired to use at least substantially translucent ferrite particles that have high magnetic permeability. Translucency is desirable so that light can shine through the lotion and magnetic permeability is desirable so that detection is enabled. Detection involves looking for particular characteristics upon an impedance curve that indicate the presence of the desired multi-phase topical. In one embodiment, a ferrite is packaged into spheres ranging from about 0.5 μm to about 40 μm of outer diameter, is substantially translucent, and is coated with a layer of translucent biocompatible plastic such that the ratio of outer and inner diameters of the shell does not exceed approximately 2.0 (exemplary materials of the shell include, for example, acrylic and polycarbonate) and is dispersed into a non-conductive oily matrix. A suitable non-conductive oily matrix includes Versigel lotion. Suitable ferrites that may be employed in such a multi-phase system include, for example, Fe₃O₄, BaFe₁₂0₁₉, Rb₂CrCl₄, and Fe:ZnO.

It is desirable that the compound encapsulated within the particles have magnetic permeability, because when you apply an external magnetic field the domains within the compound may be oriented in a desirable manner along the force lines of the magnetic field. Dispersing such encapsulated compound(s) in a lotion can create a unique electromagnetic signature that allows the compound(s) to uniquely identify the presence and/or the absence of lotion (e.g., in a particular region of skin tissue).

In one embodiment, the lotion including encapsulated compounds dispersed therein is applied to a part of a body to be treated. An electromagnetic radiation force is applied in the region of the applied lotion. Any change in the electromagnetic radiation of the lotion is detected. If the expected change due to the encapsulated compounds is observed then the lotion is detected as present on the part of the body and the device is allowed to fire.

In another embodiment of a multi-phase system, several layers of markers are each encased into one another like a core with layers, and one or more of the various layers provide a unique interrogative signal signature (e.g., a unique impedance spectrum). In this way, a particle itself can act as a tag or identifier much like an RFID device. Optionally, the particle can have an expiration date encoded into it.

The same layering concept (e.g., core with various layers) can be used with fluorescence whereby one or more shells of fluorescent material can be layered over a core thereby to modify the fluorescent spectrum to make it more distinct than the fluorescent spectrum of just a solution. Suitable shell layers can include eosin (a FDA approved fluorescent dye for drug and cosmetics that has high fluorescence). One problem is that eosin is an ingredient used in lipstick so if eosin were used as a marker, the contrast risks being low where subjects have eosin from lipstick or other cosmetics on their skin surface. Encapsulating layers of eosin in a sphere could enable modification of its fluoresce to ensure it has a profile that is distinct from lipstick or other cosmetics.

Optionally, a layered particle may be disposed with one or more layers of markers that impact the impedance spectrum and one or more layers of markers that impact the fluorescence. In another multi-phase system, encapsulated particles that impact impedance are suspended together with encapsulated particles that impact fluorescence.

Suitable lotions and/or topicals can take the form of a gel, polymer film, or ointment. A polymer film can be impregnated with an identifier that demarcates the area to be treated (e.g., shaped to surround the lip or the eye). In one embodiment, a lotion or film can be designed to prevent overtreatment of a skin surface area by being made sensitive of the fact that it has previously been treated. For example, the lotion or the film can have a marker that changes (e.g., degrades) after exposure to directed energy to protect the consumer from overtreatment (e.g., multiple treatments). In such an embodiment, for example, the required marker level will have changed after one or more treatments such that further treatment of a previously treated area will be prevented when the sensor fails to recognize the required level to enable treatment (e.g., the level required to allow firing is no longer present in the region that was previously treated due to degredation).

In some embodiments, the device can recognize the lotion by any of the above described properties/markers. Where light energy (e.g., laser energy) is used as directed energy for treatment, eye protection becomes paramount. Optionally, the lotion may contain a mild eye irritant to deter the user from applying the lotion to the eye and, thus, to lessen the likelihood of direct treatment into the eye.

The device should be able to distinguish between lotion alone and lotion on the skin. This capacity will avoid a person putting a blob of the lotion onto the tip of the device to enable the energy source to fire. One way to ensure such a distinction is made by the device is to make the skin be a part of the differential measurement. The techniques described in association with FIGS. 7a, 7b, 8a, and 8c are sensitive enough to detect BU where there is bulk unlimited lotion (a layer of lotion measuring at least 5 mm thick on the device), but there is no contact of the device with the skin.

In some embodiments, systems for detecting proximity of human skin include a topical marker compound with electrical properties formulated in such a way that electrical properties of the compound applied to skin are substantially different from both electrical properties of skin alone and electrical properties of the bulk compound alone. Devices for measuring the difference in the electrical properties of compound alone, skin alone, or compound on skin include, for example, impedance sensors. Methods of detecting the proximity of human skin, include applying a compound formulated with electrical properties to the skin and using a device, such as an impedance sensor, to interrogate the area of interest.

In one embodiment, in the first interrogation step, the presence of the marker compound is established with certainty (differentiation mode). In the second interrogation step, contact with skin is established with certainty (uniformity mode). In some embodiments, during the interrogation step, a common mode rejection measurement scheme is used. A device for measuring the difference in electrical impedance may be employed to interrogate the subject's tissue (e.g., skin). The device may have multiple interrogation point(s). Suitable topical compounds, including, for example, multi-phase compounds, have electrical properties in the uniformity mode that are in the range of P₁>2000Ω, 0.1<P₂<0.75, and 1.1<P₃<2. Suitable topical compounds, including, for example, multi-phase compounds, have electrical properties in the differentiation mode that are in the range of 0<P₁<1750Ω, 0.2<P₂<0.59, and 1.25<P₃<1.59. A multi-phase system can utilize the same interrogation techniques described in association with FIGS. 1-8, but with a suspension in the topical that provides a marker. The parameters P₁, P₂, and P₃ are exemplary and the parameter values can vary based on the modulation frequency or frequencies that are employed.

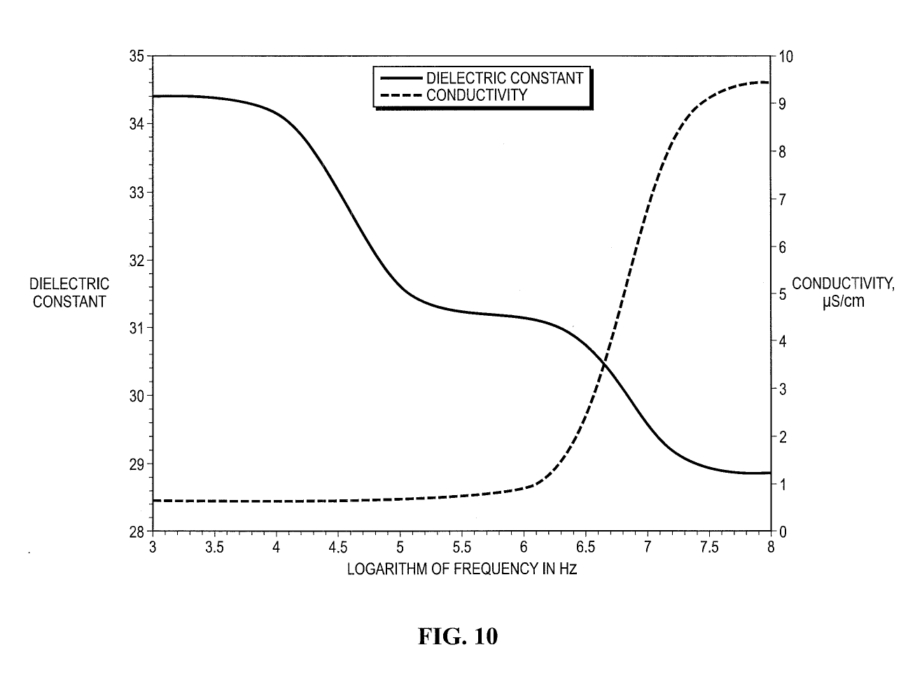

FIGS. 9 to 14 illustrate an exemplary embodiment of a multi-phase conductive/dielectric system that creates unique impedance characteristics. A multi-phase conductive/dielectric system can make the lotion truly unique and robust to avoid a false “key” detection that begins the cycle that enables the device to fire as compared, for example, to the above-described impedance detection/differentiation system. FIG. 9 is a schematic representation of a spherical particle containing a conductive compound (electrolyte), covered with a non-conductive shell and suspended in a surrounding ambient medium. The following parameters are used in one example:
1. Particle diameter (D): 30 μm
2. Shell thickness (d): 2 μm.
3. Shell material: polystyrene has dielectric constant (εₛ).
4. Material inside the particle: 1M (mole/liter) solution of KCl in water has dielectric constant (εᵢ).
5. Ambient material: glycerol, 10% water (low conductivity matrix) has dielectric constant (εₐ).
6. Particle concentration: 3.5·10⁷ cm⁻³ (volume fraction 49.5%).
7. (ε_{q}) would be the effective dielectric constant of an equivalent homogeneous medium that lacks particles, however, ε_{q} is frequency dependent whereas in a real multi-phase solution the properties are constant. FIG. 10 depicts ε_{q} as the solid line Dielectric constant.

The dielectric properties of the three components of the suspension (e.g., the polystyrene, the KCl and the glycerol) are considered to be frequency independent.

The spherical particles of the multi-phase system may have parameters that fall within the following ranges: the particle diameter (D) can have an outer diameter range of from about 0.5 to about 40 μm; the shell thickness (d) can have a range of from about 0.1 μm to about 20 μm; the shell material has a relatively low electrical conductivity (e.g., a conductivity that is <10⁻⁶ S/m), the shell material has a relatively low dielectric constant (e.g., a dielectric constant <50) and the shell material has no solvent permeability; the material inside the particle can be a relatively low concentration solution of NaCl and/or KCl in water and will be used as an electrolyte (1-50 mg/L salt in DI water); alternatively, a ferrite material is used to fill the particle shell; the ambient material provides a low conductivity matrix and is any oil such as glycerol in 10% water; the particle concentration has a volume fraction that ranges from about 0.1% to about 60%.

FIG. 10 shows the effective dielectric properties (e.g., the dielectric constant) of the multi-phase system (e.g., suspension) on the y-axis versus the logarithm of frequency in Hertz on the x-axis. The effective dielectric properties of the suspension (dielectric constant ε_{q} is depicted by the solid line, the conductivity is depicted by the dashed line) depend on the modulation frequency as shown in FIG. 10. The impedance sensor sends modulated voltage to the system and then measures the current that results from the modulated voltage. The modulated frequency ranges from about 0 to about 100 MHz, or from about 0 to about 1 GHz. Using the current and the voltage information the complex impedance value at the modulation frequencies is deduced using Ohm's law.

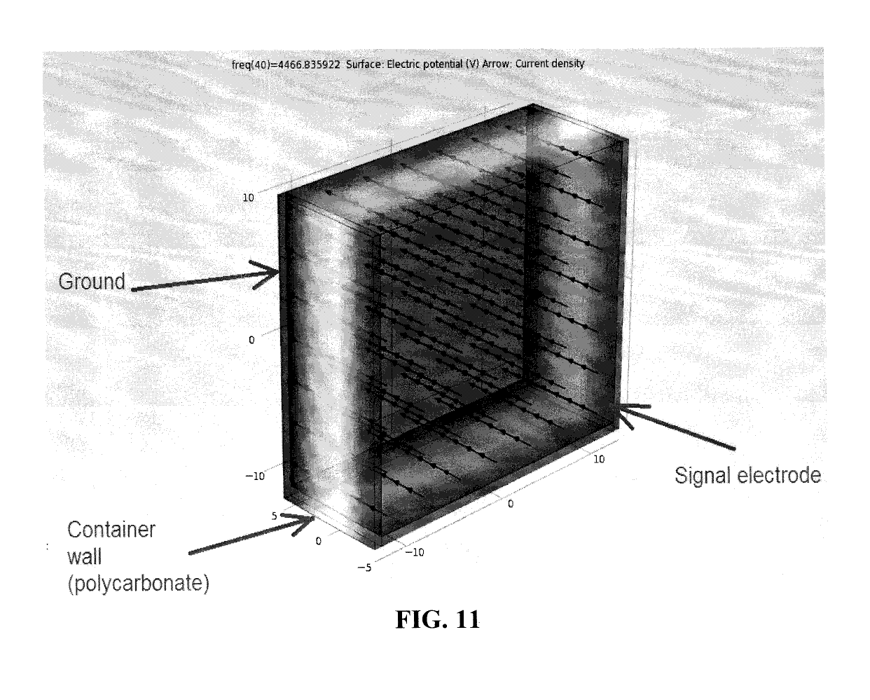

FIG. 11 depicts an experimental configuration that is employed to measure the impedance spectrum of a lotion. Here the experimental configuration shows the capacitor geometry with the capacitor having non-conductive walls (edges). The impedance spectrum of any lotion may be evaluated using such an experimental configuration and a Matcad model. The capacitor geometry in the example shown in FIG. 11 has the following parameters:
1. Plate size (height×width): 21×25 mm.
2. Distance between the plates: 10 mm

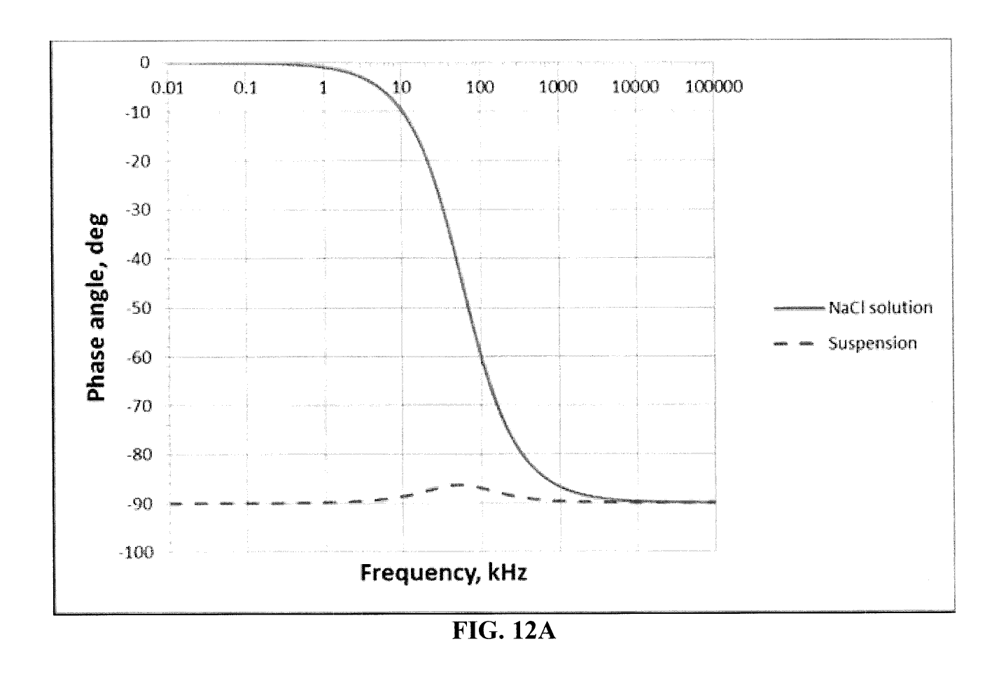

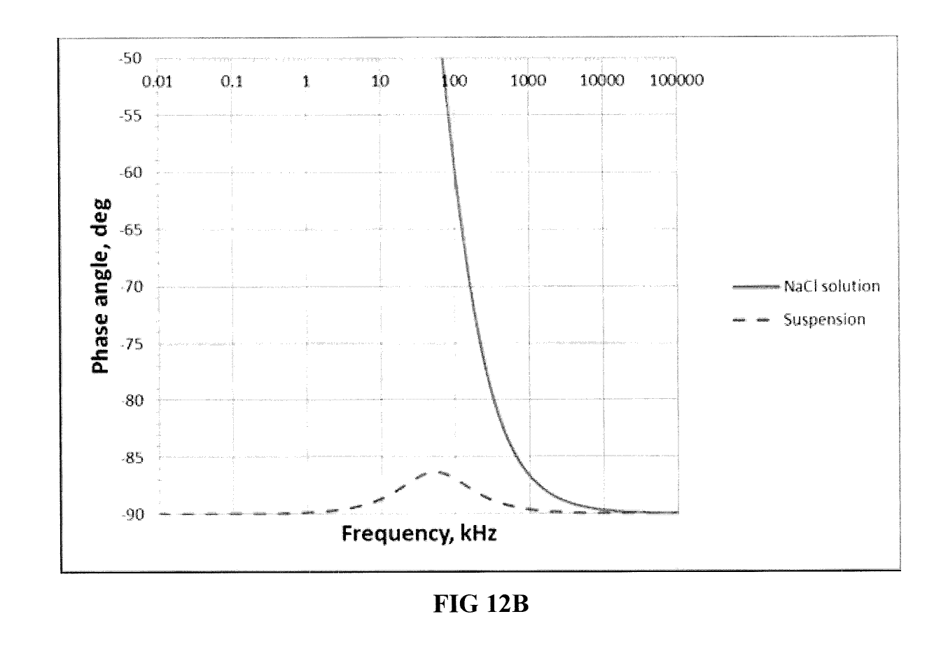

FIGS. 12a and 12b show a predicted spectrum of the impedance phase angle (e.g., a model) resulting from an example of a multi-phase system evaluated using capacitor geometry shown in FIG. 11. More particularly, FIGS. 12a and 12b show the phase shift of the exemplary homogeneous medium (solid line) and that of an exemplary multi-phase system (dashed line) in capacitor geometry vs. frequency. FIG. 12a shows the phase shift for a full range of angels. FIG. 12b zoom into the range of angles to show the phase shift for from about −50 to about −90 degrees range. The spectrum of FIGS. 12a and 12b demonstrates resonance features in the phase angle spectrum of the multi-phase system (dashed line) that enable identification of the multi-phase lotion (in this embodiment the lotion is identified by the peak in the phase angle of the impedance curve). The formulation parameters for the multi-phase system modeled in FIGS. 12a and 12b include:

A spherical shell with outer diameter of about 30 microns and shell wall thickness of about 2 microns, a shell material having zero conductivity and a dielectric constant of 3, the shell is filled with an aqueous solution of NaCl having a concentration of about 20 μM, the volume fraction of the particles is about 20%, and the surrounding medium has zero conductivity and a dielectric constant of 4. The homogeneous medium simulated was an aqeous NaCl solution having a concentration of about 20 μM.

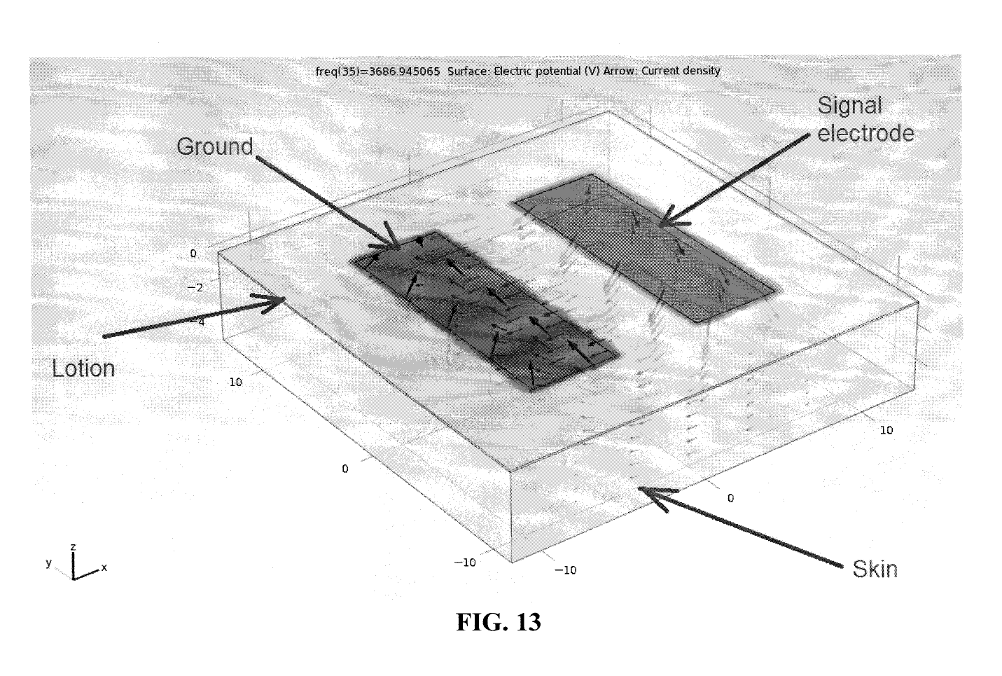

FIG. 13 shows the layout of a skin sensor model. This model shows variable thickness of the topical (e.g., the lotion layer) layer 200 disposed on top of the skin 250. A signal electrode 310 and a ground 320 are placed on the surface of the topical layer 200. The impedance properties of the topical layer 200 and the skin 250 are evaluated with this skin sensor configuration (FIG. 13) using an FEM model (a finite element model) that had the following parameters:
1. Electrodes (i.e., the signal electrode and the ground) are modeled as equipotential surfaces.
2. Electrodes are sized: 15 mm×5 mm.
3. Electrode separation (between the centers): 10 mm.
4. Thickness of lotion (e.g., the topical layer 200 thickness): 0, 100 μm, 200 μm.
5. Frequency range: 1 kHz-100 MHz.

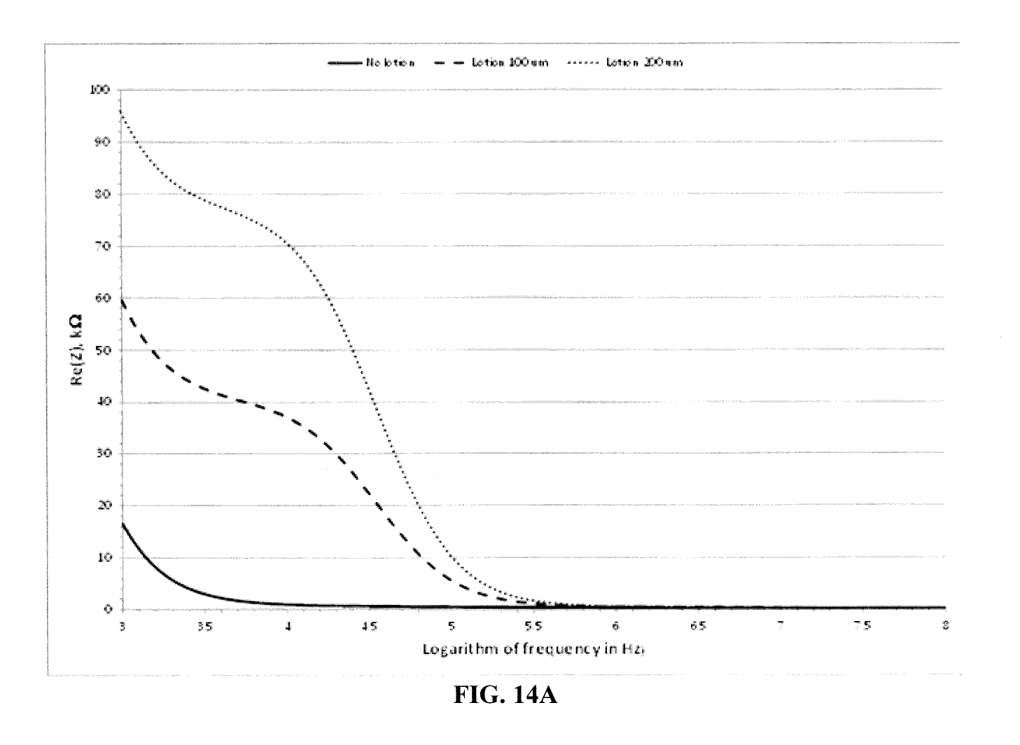

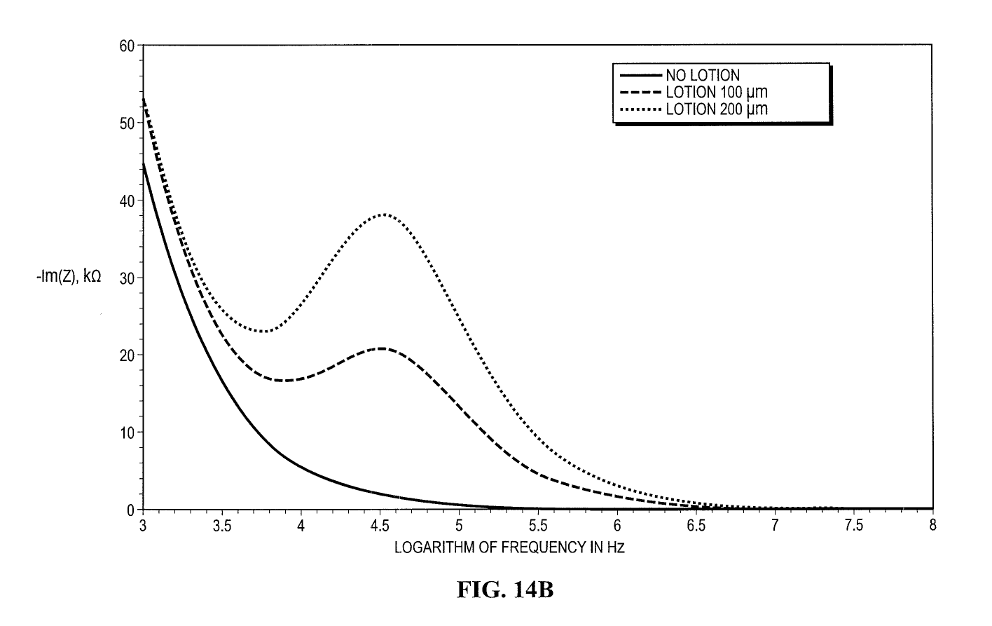

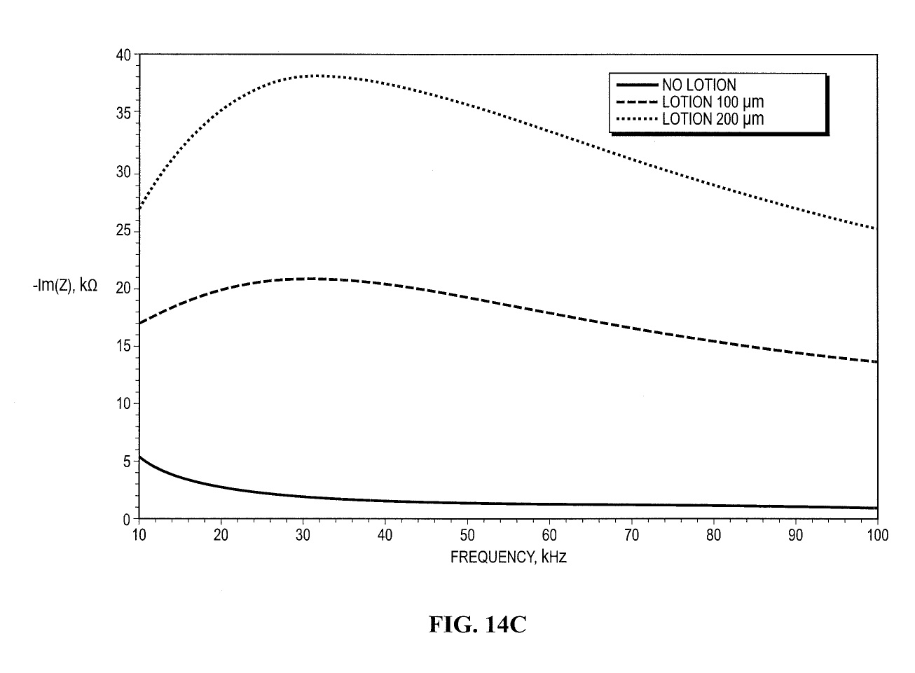

The results are shown in FIGS. 14a, 14b and 14c. The resonance properties of the multi-phase system are maintained in the sensor geometry and allow reliable differentiation between the situation of bare skin with no lotion (solid line) vs. the lotion-on-skin at 100 μm thickness, e.g., thin layer, (dashed line) vs. lotion-on-skin at 200 μm thickness, e.g., thick layer, (dotted line).

FIG. 14a shows the real part of the impedance of the exemplary multi-phase system vs. frequency for the skin sensor model (logarithmic scale) under conditions of bare skin with no lotion (solid line) vs. the lotion-on-skin at 100 μm thickness (dashed line) vs. lotion-on-skin at 200 μm thickness (dotted line). The real part of the impedance in the multi-phase system enables identification of the multi-phase lotion in both the cases of lotion-on-skin at 100 μm thickness (dashed line) vs. and lotion-on-skin at 200 μm thickness (dotted line).

FIG. 14b shows the imaginary part of the impedance of the exemplary multi-phase system vs. frequency for the skin sensor model (logarithmic scale) under conditions of bare skin with no lotion (solid line) vs. the lotion-on-skin at 100 μm thickness (dashed line) vs. lotion-on-skin at 200 μm thickness (dotted line). The imaginary part of the impedance in the multi-phase system enables identification of the multi-phase lotion in both the cases of lotion-on-skin at 100 μm thickness (dashed line) vs. and lotion-on-skin at 200 μm thickness (dotted line).

FIG. 14c shows the imaginary part of the impedance of the exemplary multi-phase system vs. frequency for the skin sensor model (linear scale) under conditions of bare skin with no lotion (solid line) vs. the lotion-on-skin at 100 μm thickness (dashed line) vs. lotion-on-skin at 200 μm thickness (dotted line).

Review of FIGS. 14a and 14b reveals curves that have characteristic features (e.g., a bump) on FIG. 14a at around the logarithm of 4 Hz (about 10 kHz) and in FIG. 14b at around the logarithm of 4.7 Hz (about 30 kHz). The characteristic features indicate the presence of a multi-phase system impedance versus the control of no lotion and it would also show such a differentiation versus a control lotion that does not have impedance value impacted by a multi-phase system.

1. A photocosmetic device, comprising:
a handpiece including a source for generating energy for application to tissue, said handpiece having a distal end through which the energy can be applied to tissue,
at least a sensor coupled to the handpiece and adapted to generate a signal in response to detecting contact between at least a portion of said distal end and a topical substance disposed over a portion of the tissue, and
a feedback mechanism in communication with the sensor and the energy source for activating said source in response to receiving said detection signal.

2. The photocosmetic device of claim 1, wherein said feedback mechanism is adapted to deactivate said energy source subsequent to its activation in response to absence of said detection signal from the sensor.

3. The photocosmetic device of claim 1, wherein said handpiece further includes a scan mechanism for directing said energy to different portions of the tissue.

4. The photocosmetic device of claim 2, wherein said feedback mechanism is in communication with said scan mechanism so as to activate said scan mechanism in response to receiving said detection signal from the sensor.

5. The photocosmetic device of claim 3, wherein said scan mechanism is adapted to direct the radiation to said different tissue portions subsequent to its initial activation based on a predetermined protocol.

6. The photocosmetic device of claim 1, further comprising a mechanism to differentiate between signals from a topical substance disposed over a portion of the tissue and from a bulk volume of the topical substance.

7. A photocosmetic device, comprising
a handpiece including a source for generating energy for application to tissue;
at least one detector coupled to the handpiece for detecting a topical substance disposed on the tissue when placed in proximity of the topical substance; and
a feedback mechanism in communication with the source and the detector, said feedback mechanism activating said source in response to detection of said topical substance on the tissue by the detector.

8. The photocosmetic device of claim 7, wherein said feedback mechanism is adapted to deactivate said source subsequent to activation in response to a signal from the detector indicating absence of said topical substance on the tissue.

9. A photocosmetic device, comprising
a source for generating electromagnetic radiation,
a radiation transmission path for transmitting the radiation from the source to a radiation transmissive optical window through which the radiation can be applied to the skin, said optical window having a perimeter adapted for positioning over the skin,
a plurality of sensors for detecting presence of a topical substance over the skin, said sensors being positioned relative to the window such that each sensor is capable of determining whether a selected portion of said perimeter is in contact with or in proximity to the topical substance disposed over the skin,
a feedback mechanism in communication with the sensors and the radiation source, said feedback mechanism deactivating said radiation source if at least one of said sensors indicates absence of contact between a respective portion of said perimeter and the topical substance disposed over the skin.

10. A photocosmetic device, comprising
a handpiece adapted for positioning in proximity of tissue at a distal end thereof, said handpiece comprising:
an optical path for transmitting energy from an energy source to said distal end for application to tissue,
at least a sensor coupled to said handpiece for generating a signal indicative of presence of a selected topical substance on the tissue in proximity of said distal end, and
a feedback mechanism in communication with said sensor and said source, said feedback mechanism activating said source in response to receiving said signal from the sensor.

11. The photocosmetic device of claim 10, said feedback mechanism is adapted to deactivate said source subsequent to its activation in absence of said signal from the sensor.

12. A photocosmetic device, comprising:
a frame adapted for positioning in proximity of tissue to define an area of the tissue;
a source for generating optical energy;
a scan mechanism coupled to said source for moving said source so as to apply optical energy to different portions of said area of the tissue;
a sensor adapted for detecting presence of a topical lotion on the tissue, said sensor generating a signal in response to detection of the topical lotion on the tissue; and
a feedback mechanism in communication with said scan mechanism and said sensor, wherein said feedback mechanism triggers said scan mechanism to effect the movement of said source in response to receiving said signal from the sensor.

13. A photocosmetic device adapted for application of optical energy to tissue, said device comprising:
a sensor adapted for detecting a topical substance in contact with the tissue; and
a control mechanism in communication with said sensor, wherein said control mechanism permits application of the optical energy to a tissue portion only if the sensor detects said topical substance on said tissue portion.

14. The photocosmetic device of claim 13, further comprising a source for generating said optical energy.

15. The photocosmetic device of claim 14, wherein said control mechanism causes a transition of said source from a de-activated state to an activated state in response to detection of said topical substance on the tissue by said sensor.

16. The photocosmetic device of claim 15, wherein said control mechanism maintains said source in an activated state subsequent to its initial activation if the sensor continues to detect said topical substance on the tissue.

17. A system for treating tissue, comprising:
a handpiece having an energy source configured to deliver energy to a tissue surface;
a topical substance configured to be applied to the tissue surface; and
a recognition mechanism in communication with the energy source, and configured to allow activation of the energy source in response to recognition of said topical substance on at least one recognition site of the tissue surface.

18. The system of claim 17, wherein the topical substance includes at least one tag defining at least one characteristic configured to be recognized by the recognition mechanism.

19. The system of claim 18, wherein the recognition mechanism is configured to distinguish the topical substance having the at least one tag from another topical substance having the at least one tag.

20. The system of claim 18, wherein the recognition mechanism is configured to distinguish the topical substance having the at least one tag from the topical substance having at least one alternative tag.

21. The system of claim 17, wherein the recognition mechanism includes a sensor in communication with the at least one recognition site of the tissue surface.

22. The system of claim 21, wherein the sensor is coupled to the handpiece.

23. The system of claim 21, wherein the senor is coupled to the energy emitter.

24. The system of claim 21, wherein the sensor includes a light emitter and a detector.

25. The system of claim 24, wherein the sensor is configured to determine a parameter of the recognition site determinative of a presence or absence of the topical substance.

26. The system of claim 17, wherein the topical substance is a multi-phase system, at least a first and a second phase of the multi-phase system each contributing to a signal indicative of the presence of the topical substance.

27. The system of claim 26, wherein the first phase is a background solution, and the second phase is at least one particle suspended in the background solution.

28. The system of claim 26, wherein the multi-phase system includes two or more distinct active components.

29. The system of claim 26, wherein the multi-phase system includes conductive particles suspended in a dielectric solution.

30. The system of claim 29, wherein the multi-phase system includes a ferromagnetic substance suspended in a dielectric solution.

31. The system of claim 26, wherein the multi-phase system includes at least one layered tag, at least one of the layers providing a unique interrogative signal signature.

32. The system of claim 31, wherein the tag is utilized as an identifier.

33. The system of claim 17, wherein the topical substance is a ferromagnetic substance.

34. A system for treating skin, comprising:
a device having a frame and an energy source movably coupled to the frame;
a sensor coupled to the device, and sized and configured to be positioned into communication with a treatment site, and further configured to determine if a topical substance having a desired characteristic is applied to the treatment site; and
a mechanism for deactivating the energy emitter if the sensor senses absence of the topical substance at the treatment site.

35. The system of claim 34, wherein the sensor is coupled to the energy emitter.

36. The system of claim 34, wherein the sensor is coupled to the frame.

37. The system of claim 36, wherein a plurality of sensors are coupled to the frame.

38. The system of claim 34, wherein the sensor is an impedance sensor.

39. The system of claim 34, wherein the sensor is configured to distinguish between the topical substance having the desired characteristic and another topical substance not having the desired characteristic.

40. The system of claim 34, wherein the sensor is configured to determine if the topical substance having the desired characteristic has expired.

41. The system of claim 34, wherein the energy emitter is configured to deliver a desired treatment protocol configured to effect a treatment for a condition.

42. The system of claim 41, wherein the condition is acne, unwanted hair, wrinkles, lesions, vascular lesions, or cellulite.

43. A skin treatment system, comprising:
a device configured deliver a therapeutically effective amount of energy to an area of skin;
a topical substance configured to be applied to a patient's skin, and further configured to have a desired characteristic indicative of an identity of the topical substance;
a sensor coupled to the device, and configured to detect the characteristic of the topical substance; and
a mechanism for activating the device only if the sensor detects the characteristic of the topical substance.

44. The system of claim 43, wherein the topical substance includes at least one tag configured to exhibit the characteristic of the topical substance.

45. The system of claim 44, wherein the topical substance includes a plurality of tags configured to exhibit the characteristic of the topical substance.

46. The system of claim 43, wherein the sensor is configured to distinguish the topical substance applied to skin and the topical substance not applied to skin.

47. The system of claim 43, wherein the topical substance includes at least one tag configured to indicate an expiration date of the topical substance.

48. The system of claim 47, wherein the mechanism is configured to de-activate the device if the topical substance is expired based on the expiration date.

49. A topical substance for applying to a tissue surface, comprising:
a liquid solution; and
at least one tag dispersed in the liquid solution, the at least one tag configured to be identifiable by a sensor.

50. A method of initiating tissue treatment, comprising:
positioning a sensor in communication with a recognition site of a tissue surface;
analyzing the recognition site with the sensor to determine if a desired topical substance is present at the recognition site; and
activating an energy emitter to deliver energy to a treatment site only if the topical substance is present at the recognition site.

51. The method of claim 50, wherein the recognition site is representative of a larger treatment site.

52. The method of claim 50, wherein the recognition site is the treatment site.

53. The method of claim 50, further comprising repeating the activating step so as to treat multiple treatment sites.

54. The method of claim 53, wherein the analyzing step is performed prior to each activating step.

55. The method of claim 53, wherein the analyzing step is performed only prior to the first activating step.

56. A method of initiating tissue treatment, comprising:
providing a treatment device having an energy emitter at least partially disposed within a handpiece, the device configured to detect a detectable characteristic of a topical substance applied to a tissue site;
analyzing the tissue site to detect if the topical substance is present at the tissue site; and
activating the energy emitter only if the topical substance is present at the tissue site.
